(19) United States
(12) Patent Application Publication
(10) Pub. No.: US 2026/0357269 A1
(43) Pub. Date: Nov. 12, 2026

(54) COMPOSITIONS AND METHODS FOR MODULATING COGNITION WITH INTERFERING RNA

(71) Applicants: John Lawrence MEE, Stuart, FL (US); Dean RADIN, Stuart, FL (US); Troy T. ROHN, Stuart, FL (US)

(72) Inventors: John Lawrence MEE, Stuart, FL (US); Dean RADIN, Stuart, FL (US); Troy T. ROHN, Stuart, FL (US); Jim FALLON, Stuart, FL (US)

(21) Appl. No.: 19/488,333

(22) PCT Filed: May 24, 2024
(86) PCT No.: PCT/US2024/031049

§ 371 Date: Nov. 26, 2025

Related U.S. Application Data
(60) Provisional application No. 63/470,150, filed on May 31, 2023.

(51) Int. Cl. C12N 15/113; A61K 9/00; A61K 31/713; A61K 48/00; C12N 15/86

(52) U.S. Cl. CPC C12N 15/1138; A61K 9/0043; A61K 31/713; A61K 48/0058; C12N 15/86; C12N 2310/14; C12N 2320/32; C12N 2750/14143

(57) ABSTRACT

Various aspects of this disclosure relate to interfering RNA and methods of treating patients that present with mental health conditions by administering AAV9 vectors that encode an interfering RNA that reduces translation of a human neurotransmitter receptor in neurons. The AAV9 vectors may be administered intranasally.

### CROSS-REFERENCE TO RELATED APPLICATION

This is an International Patent application claiming priority to and the benefit of U.S. Provisional Patent Application No. 63/470,150, filed 31 May 2023, which is incorporated by reference herein in its entirety.

### TECHNICAL FIELD

The present disclosure relates generally to systems and methods for improving mental health and/or performance using an intranasally-administered nucleic acid that encodes an shRNA.

### BACKGROUND

Depression and anxiety affect about 3.5 percent of the global population. The anxiolytic and antidepressant drug market relies mainly on pharmaceutical products that are over 30 years old, which presents opportunities for innovation. The efficacy of current selective serotonin reuptake inhibitor (SSRI) medications, such as Prozac®, Celexa®, and Zoloft®, are limited, and these pharmaceuticals display serious side effects. A demand exists for new and improved interventions that treat anxiety and depression more effectively, which presents opportunities for disruption and innovation in the market.

Research demonstrates that 30 to 50 percent of patients with anxiety disorder or major depressive disorder (MDD) do not respond adequately using SSRIs. Additionally, patients with anxiety and depressive disorders can experience relapse rates of 50 to 80 percent over several years depending on their treatments. Long-term benzodiazepine use can cause drug dependence, and the side effects of SSRIs include sexual dysfunction, chronic nausea, compromised digestive function, dry mouth, weight gain, sleep disturbances, and, in serious cases, suicide. Innovative interventions that provide sustained improvement of symptoms of depression and anxiety and that minimize adverse events would have tremendous value.

### SUMMARY

One aspect of the present disclosure includes methods of treating a patient with a mental health condition. These methods include administering a dose of a vector that can mediate transcription of an interfering RNA such as a short hairpin RNA (shRNA). Such methods may also include administering a brain region activator to activate a target brain region for treatment. The methods further include transporting the vector to active neurons within the target brain region using hemodynamics. The vectors may then modulate brain activity within the target brain region.

Another aspect of the present disclosure relates to methods of improving a patient's cognitive capacity. Such methods include administering a dose of a vector that can mediate transcription of an shRNA. These methods also include administering a brain region activator to activate a target brain region. The methods further include transporting the vector to the active neurons within the target brain region using hemodynamics. The vector may then improve and/or enhance brain activity within the target brain region.

The foregoing broadly outlines several features and technical advantages of examples of this disclosure so that the detailed description that follows may be better understood. Additional features and advantages will be described hereinafter. The concepts and specific examples disclosed herein may be readily utilized as a basis for modifying or designing other methods, constructs, and/or systems for carrying out the same or similar purposes of the present disclosure. Such equivalent constructions do not depart from the spirit and scope of the appended claims. Features that are believed to be characteristic of the concepts disclosed herein, both as to their organization and method of operation, together with associated advantages will be better understood from the following description when considered in connection with the accompanying figures. Each of the figures is provided for the purpose of illustration and description only, and not to define limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 is a diagram listing a variety of biologics in accordance with the principles of the present disclosure.

FIG. 2 is a diagram listing a variety of brain region activators in accordance with the principles of the present disclosure.

FIGS. 3A, 3B, 4A, and 4B illustrate an exemplary method of delivering a neuron-editing biologic drug intranasally in accordance with the present disclosure.

FIG. 5 is a map of plasmid pAAV9-Mecp2-spCas9, which is used to express a nucleic acid that encodes spCas9 with inverted terminal repeats (ITRs) for packaging the nucleic acid into an adeno-associated virus (AAV) vector.

FIG. 6 is a histogram that depicts a statistically significant 8.47-fold decrease in HTR2A gene expression (which encodes 5HT-2A receptor) in brains of mice treated with AAV9 vectors to knockout 5HT-2A receptor.

FIG. 7 is a fluorescence microscopy image that depicts the expression of GFP throughout the CNS including olfactory bulb (OB), cortex, and sub-cortical areas including the interpeduncular nucleus (IPN) for a mouse treated with AAV9 vectors to knockout 5HT-2A receptor.

FIG. 8 is a histogram that depicts the quantification of anti-5HT-2A receptor immunohistochemistry-associated fluorescence in brains of mice treated with AAV9 vectors to knockout 5HT-2A receptor and in brains of vehicle-control mice.

FIG. 9 contains fluorescence microscopy images of the cortex, the Purkinje cell layer of the cerebellum, and pontine gray nuclei for mice treated with AAV9 vectors to knockout 5HT-2A receptor and corresponding images for vehicle-control mice. The left panels show fluorescence associated with GFP, which demonstrates transfection with the AAV9 vector that also encodes guide RNA (gRNA). The right panels depict fluorescence associated with 5HT-2A receptor. The nuclear stain DAPI is depicted in gray whereas GFP and 5HT-2A associated fluorescence is depicted in white. Mice that received AAV9 vectors to knockout 5HT-2A receptor displayed increased fluorescence associated with GFP and decreased fluorescence associated with 5HT-2A receptor relative to control in each set of images.

FIG. 10A-10C are graphs for mice subject to a marble-burying test. FIGS. 10A and 10B show statistically significant decreases in number of marbles buried by mice that received AAV9 vectors to knockout 5HT-2A receptor relative to vehicle control mice at 5-weeks and 7-weeks post-AAV9 treatment, respectively. FIG. 10C demonstrates that the treated and control mice displayed no significant difference in body weight although the knockout mice trended toward lower bodyweights.

FIG. 11A-11E are graphs for mice subject to a light/dark behavioral test. FIGS. 11A, 11B, and 11C show that mice treated with AAV9 vectors to knockout 5HT-2A receptor displayed a statistically significant increase in time spent in the lit box, entries into the lit box, and locomotion in the lit box, respectively, relative to vehicle-only controls. FIG. 11D shows that mice treated with AAV9 vectors to knockout 5HT-2A receptor displayed a similar amount of time spent in the lit box relative to positive-control mice that received diazepam.

FIG. 11E depicts the number of entries into the lit box relative for AAV9-treated mice relative to positive-control mice that received diazepam, which indicates that the AAV9-treated mice exited the lit box less frequently than diazepam-treated mice.

FIG. 12 is a graph for mice subject to an elevated plus maze (EPM) test and depicts a statistically significant decrease in entries into the closed arm of the EPM for mice treated with AAV9 vectors to knockout 5HT-2A receptor relative to vehicle controls.

FIG. 13 is a graph that depicts a statistically-significant decrease in 5HT-2A receptor mRNA in the olfactory bulbs of mice treated with AAV9 vectors encoding interfering RNA verses vehicle controls (p-value=0.006 441).

FIGS. 14A, 14B, 15A, and 15B are histograms for mice subject to a light/dark behavioral test. FIGS. 14A and 14B show that mice treated with AAV9 vectors to knock down 5HT-2A receptor displayed a statistically significant increase in entries into the lit box and time spent in the lit box, respectively, at 5-weeks following treatment relative to vehicle-only controls. FIG. 15A shows that mice treated with AAV9 vectors to knock down 5HT-2A receptor displayed an increase in entries into the lit box at 8-weeks following treatment relative to vehicle-only controls; numbers of entries were not statistically significant at p-value <0.05, but the observed p-value of 0.058 coupled with 5-week data nevertheless suggests a real difference between the experimental and control groups. FIG. 15B shows that mice treated with AAV9 vectors to knock down 5HT-2A receptor displayed a statistically significant increase in time spent in the lit box at 8-weeks following treatment relative to vehicle-only controls.

FIG. 16 is a line graph of mouse bodyweight for mice treated with AAV9 vectors to knock down 5HT-2A receptor (lower body weight line) and vehicle controls (higher body weight line). Mice treated with AAV9 vectors to knock down 5HT-2A receptor displayed lower body weight than vehicle controls, but this difference was not determined to be statistically significant at p-value<0.05.

FIGS. 17A and 17B are fluorescence image overlays of human glutamatergic neurons treated with either a AAV9 vector encoding an interfering RNA to knock down 5HT-2A receptor (FIG. 17A) or a control AAV9 vector encoding a scrambled interfering RNA (FIG. 17B). In FIGS. 17A and 17B, GFP fluorescence is overlayed with fluorescence from an anti-5HT-2A immunostaining strategy. A decrease in anti-5HT-2A associated fluorescence of about 26.8 percent was observed in images of glutamatergic neurons treated with the AAV9 vector encoding the interfering RNA relative to glutamatergic neurons treated with the control AAV9 vector encoding the scrambled version of the interfering RNA.

FIG. 18 is a histogram that depicts difference in fluorescence between fluorescence images according to FIGS. 17A and 17B. A decrease in 5HT-2A fluorescence of about 26.8 percent was observed for glutamatergic neurons treated with an AAV9 vector encoding an interfering RNA relative to glutamatergic neurons treated with a control AAV9 vector encoding a scrambled version of the interfering RNA.

FIG. 19 is a map of plasmid AAV-MeCP2-GFP-mu-HTR2a-shRNAmir, which is used to propagate the nucleotide sequence that encodes an interfering RNA to knockdown mouse HTR2A expression using an AAV vector.

FIG. 20 is a map of plasmid AAV-MeCP2-GFP-hu-HTR2A-shRNAmir, which is used to propagate the nucleotide sequence that encodes an interfering RNA to knockdown human HTR2A expression using an AAV vector.

While the embodiments described herein are amenable to various modifications and alternative forms, specific embodiments have been shown by way of example in the drawings and will be described in further detail below. The exemplary embodiments described herein shall not limit the claims. Rather, the instant disclosure covers all modifications, equivalents, and alternatives falling within the scope of the appended claims.

### DETAILED DESCRIPTION

This description provides examples, and shall not limit the scope, applicability, or configuration of the inventions of this disclosure. Rather, the ensuing description will provide those skilled in the art with an enabling description for implementing embodiments of the invention. Various changes may be made in the function and arrangement of the elements.

Various embodiments may omit, substitute, and/or add various procedures or components as appropriate. For instance, the methods may be performed in an order different than that described, and various steps may be added, omitted, and/or combined. Also, aspects and elements described with respect to certain embodiments may be combined in various other embodiments. The following systems, methods, and devices may individually or collectively be components of a larger system, wherein other procedures may take precedence over or otherwise modify their application.

The detailed description of exemplary embodiments reference the accompanying drawings, which show exemplary embodiments by way of illustration. While these exemplary embodiments are described in sufficient detail to enable those skilled in the art to practice the disclosure, other embodiments may be realized, and logical changes and adaptations in design and construction may be made in accordance with this disclosure and the teachings herein without departing from the spirit and scope of the disclosure. The detailed description is thus presented for purposes of illustration only and not for purposes of limitation.

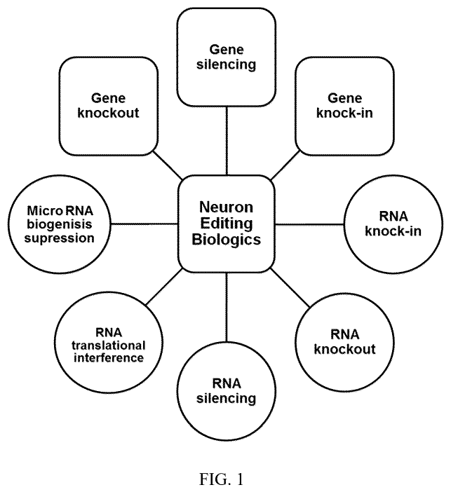

In this disclosure, biologics are biopharmaceuticals that can modify one or both of DNA and RNA of a neuron. FIG. 1 illustrates eight examples of biologics that are discussed below. These examples include RNA translational interference, which includes transcriptional engineering and editing. Transcriptional engineering and editing can interfere with translation of an mRNA into a protein, for example, by decreasing the concentration of the mRNA in a cell such that the mRNA is translated into the protein at a lower frequency relative to translation in the absence of the transcription engineering and/or editing.

In this disclosure, transcriptional engineering biologics may include RNA knockout, RNA silencing, RNA knock-in, RNA translational interference, and microRNA biogenesis suppression compounds/systems. RNA knockout compounds may include a catalytically-active RNA-editing ribonuclease complexed with a single guide RNA to alter RNA nucleotides to repress gene translation. RNA silencing compounds may include a catalytically-inactive RNA-editing ribonuclease complexed with a single guide RNA to bind to RNA nucleotides to repress or enhance gene translation. RNA knock-in biologics may include a catalytically-inactive RNA-editing ribonuclease complexed with a single guide RNA and a deaminase enzyme to cause RNA nucleobase substitutions that result in translational interference. RNA translational interference compounds may include an RNA-editing ribonuclease complexed with an RNA-expression inhibiting nucleotide and a single guide RNA to alter RNA nucleotides to cause translational interference. RNA translational interference compounds may also include interfering RNA such as short hairpin RNA (shRNA) or small interfering RNA (siRNA). MicroRNA biogenesis suppression compounds may include a catalytically-active ribonuclease complexed with a single guide RNA to alter nucleotides in biogenesis processing sites for microRNA used in the translation of genes to reduce their expression.

In this disclosure, “interfering RNA” refers to any exogenous RNA that hybridizes to a native RNA transcript when expressed in a cell to decrease the concentration of the native RNA transcript, for example, by Dicer-mediated cleavage of double-stranded RNA that comprises the exogenous RNA hybridized to the native RNA transcript. Interfering RNA includes, for example, short hairpin RNA (shRNA). “Interfering RNA” is not synonymous with small interfering RNA (siRNA), and “interfering RNA” instead encompasses siRNA as well as other RNA including, for example, shRNA.

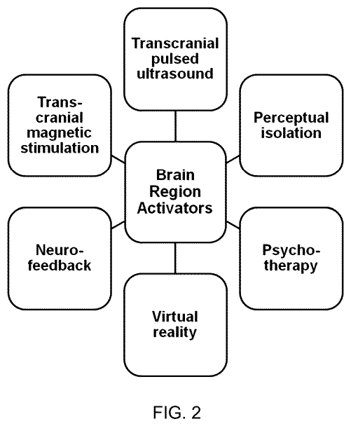

As used herein, brain region activators include methods and techniques for focusing and concentrating the neuronal activity in a patient's brain into a specific, targeted area of the brain. FIG. 2 lists six examples of brain region activators that are explained below. Others may be known to those skilled in the art.

As used herein, transcranial pulsed ultrasound is a technique that uses low-power, low-frequency ultrasound to stimulate neuron activity in the brain. It may be directed to any brain region and precisely focused to areas as small as several cubic millimeters. The power, frequency, and pulse-shape of the transcranial pulsed ultrasound are selected to activate neurons such that the neurons have an increased rate of action potentials. The transcranial pulsed ultrasound is focused on regions of the brain where uptake of a biologic is desired. Activation of neurons in the regions of the brain upon which the transcranial pulsed ultrasound of appropriately-selected power, frequency, and pulse shape is focused causes increased blood and cerebrospinal fluid to flow to the regions, which increased flow increases the probability that a biologic will contact neurons of the regions, and which therefore increases uptake of the biologic in the regions.

As used herein, transcranial magnetic stimulation is a form of neurostimulation that uses a shallow magnetic field to induce electric current to flow in small-targeted regions near the surface of the brain.

As used herein, perceptual isolation is the deliberate removal of stimuli from the senses of a subject/patient. Examples of perceptual isolation methods include sleep masks, white noise, soundproofing, and/or floatation tanks.

As used herein, neurofeedback is a type of biofeedback that measures brain waves to produce a signal that can be used as feedback to teach self-regulation of brain function. Patients can be trained to alter their brain activity to increase performance on certain tasks, which in turn changes the signal and increases the flow of blood and cerebrospinal fluid to a specified region of the brain.

As used herein, virtual reality may be an immersive, interactive, computer-generated experience that occurs in a simulated environment including auditory and visual feedback.

As used herein, psychotherapy is a wide field encompassing the use of any one or more of hundreds of different methods and techniques to improve an individual's well-being, behavior, and mental health, which includes cognitive therapy and other forms of therapies.

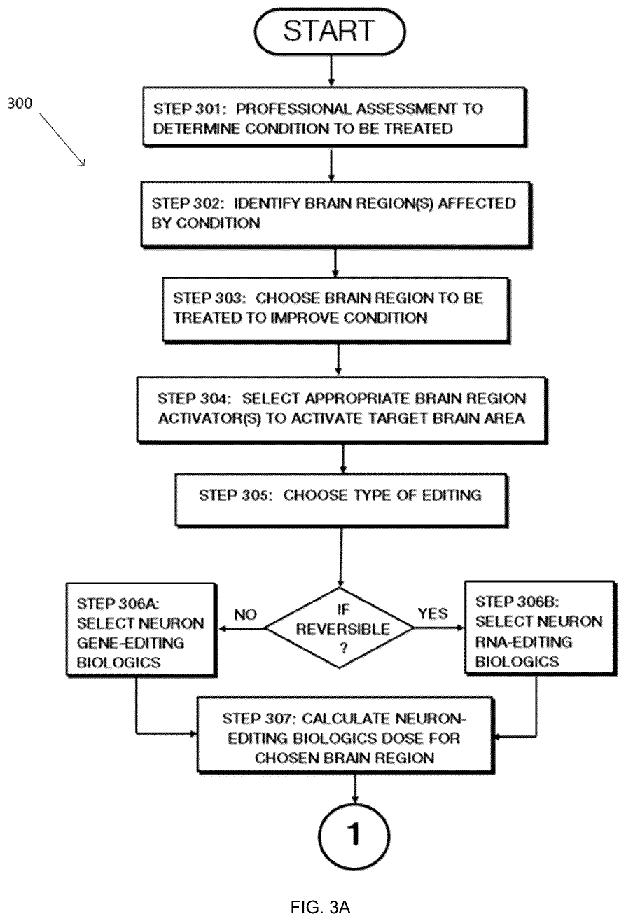

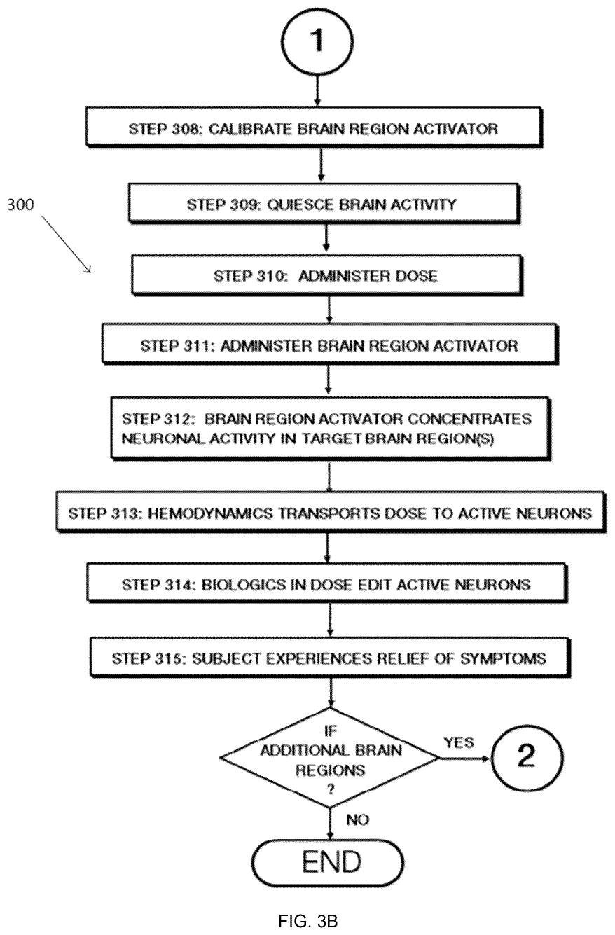

Referring next to FIGS. 3A, 3B, 4A, and 4B, a flow diagram of a method of intranasally delivering a drug is provided. Specifically, the diagram describes a method 300 for intranasally delivering gene therapies that modify neuron electrodynamic excitability in the brain to treat common mental health issues such as stress, anxiety, depression, post-traumatic stress disorder (PTSD), attention deficit disorder (ADD), and personality disorders. More specifically, as described herein, the method 300 enables a region of the patient's brain to be activated such that an intranasally-delivered drug can target the specific, activated region of the brain to treat a specific mental health condition.

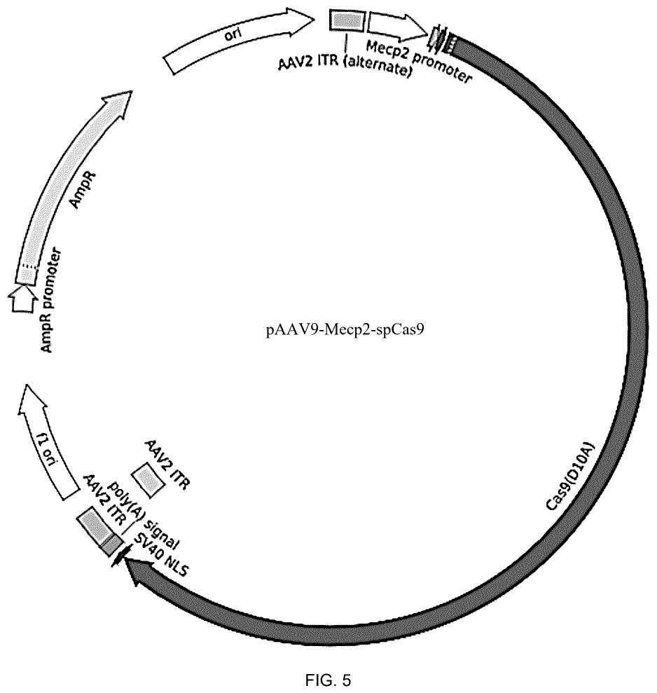

In some embodiments, intranasal delivery may allow biologics such as those disclosed herein to enter the CNS through the rostral migratory stream, which is a specialized migratory route along which neuronal precursors that originate in the subventricular zone of the brain migrate to reach the olfactory bulb. Intranasal delivery introduces biologics into the rostral migratory stream and also allows perineuronal and perivascular transit of the biologics throughout the brain. In other embodiments, intranasal delivery may facilitate delivery of the disclosed biologics along other pathways that travel in similar trajectories, including vascular, ventricular and/or neural pathways. From such pathways, the biologics may be delivered via diffusion, such as bulk diffusion, or by active uptake/pinocytosis. Regardless of the biological mechanism of transit, intranasal delivery allows the distribution of biologics throughout the entire brain, including the olfactory bulb, cortex, subcortical regions, cerebellum, and pons as shown, for example, in FIGS. 7 & 9. Intranasal delivery also allows the distribution of biologics, for example, in the thalamus, hypothalamus, hippocampus, and amygdala. Intranasal delivery allows the biologics to travel both transvascularly and transneuronally from one neuron to another.

Common mental health conditions such as stress, anxiety, depression, and inattention are all experimentally correlated with overactive neurons in specific areas of the brain. Neuron activity in these areas can be normalized through therapies that alter a neuron's structure to lower its electrical excitability. Although viral vectors and promoters can distinguish neurons from other types of cells in the body, they cannot differentiate between various regions in the brain. The illustrated method 300 allows for delivery of biologics to targeted regions and connectomes of the brain to achieve specific neurological treatment or cognitive enhancement goals.

Directing biologics to specific types of cells is known as vectoring. Current vectoring technologies can target neurons, but they cannot provide navigational specificity to individual areas within the brain. It is demonstrated herein, however, that biologics such as AAV vectors may be directed in blood and/or cerebrospinal fluid along with glucose and oxygen to specific active areas in the brain by the principles of hemodynamics.

Glucose and oxygen are the fuel which powers neuronal activity. Active neurons consume more glucose and oxygen. Unlike other cell types, neurons cannot store glucose, and so, intracellular glucose must be replenished as it is consumed. Hemodynamic principles ensure that glucose and oxygen are rapidly delivered to active neurons.

Hemodynamics principles can be used to direct AAV vectors and other biologics into active neurons that display action potentials. By employing a selective brain region activation process to concentrate the patient's neural activity into a targeted brain region, passive hemodynamics techniques can be used to draw blood and/or cerebrospinal fluid containing AAV vectors into the region. By employing a selective brain region activation process to concentrate the patient's neural activity into a targeted brain region, the method 300 can use passive hemodynamics to draw blood and/or cerebrospinal fluid containing AAV vectors into the region. Additionally, ligand vectors may be used that include targeting peptides that bind unique cell surface receptors to enable the delivery of a biologic payload to specific cell types.

A plurality of brain region activation processes can be used by cognitive neuroscience professionals to serve different applications and objectives. Method 300 outlines a framework and platform to enable cognitive neuroscience professionals to deploy a variety of applications that target specific regions of the brain to treat specific mental health conditions.

The method 300 generally includes the steps of (1) assessment of the patient/subject to identify treatable conditions and to establish goals; (2) identification of brain regions to be treated; (3) pre-treatment analysis and optional pre-treatment cognitive therapy; (4) prescription and administration of selected biologics to the patient; and (5) post-treatment follow up and optional post-treatment cognitive therapy.

In specific embodiments, the method 300 enables intranasal delivery of biologics to the olfactory epithelium with a swab, aerosol, nasal inhaler, or irrigation protocol. The olfactory bulb pathways (including the rostral migratory stream) transport the biologics directly to the limbic lobe, and the biologics target the limbic system to treat mental health conditions such as, but not limited to, inattention, stress, anxiety, and depression. In some instances, the method 300 may be used to attenuate hyperactive or hypoactive neurons in the limbic system that are responsible for inattention (ventral posterior cingulate cortex), stress (amygdala, hippocampus), anxiety (amygdala), and depression (amygdala, hippocampus).

Advantages of method 300 include (1) limiting biologic delivery to the region of interest to minimize dosing requirements and avoid affecting other areas of the brain; (2) avoid damage to biologic agents by avoiding transit that would expose them to digestive enzymes or immune system response by delivering them intranasally; (3) alleviating hippocampal and amygdaloid hyperactivity or hypoactivity that may also improve memory and could potentially halt the progression of dementia and extend longevity; and (4) in addition to one-time treatments, some applications may involve two or more iterations of treatments. For example, an AAV that encodes an interfering RNA may be readministered periodically such as two or more times per year or annually. AAV vectors have been used to transfect neurons in vivo, but mammals develop an immune response to AAV vectors, which limits the efficacy of subsequent doses. However, using viral vectors for multiple iterations is feasible with intranasal delivery because intranasal delivery transports gene editing payloads directly into the brain while avoiding an immune system response.

Once inside the brain, the method 300 includes three further methods to direct biologics to a target region of the patient's brain. First, actively-directed hemodynamic vectoring can transport the biologics to specific areas of the patient's brain that display increased rates of action potentials that are produced with brain region activators. Second, passive hemodynamic vectoring will naturally transport the biologics to the most active neurons of the patient's brain. Finally, ligand vectors that include targeting peptides designed to direct biologics to specific neuron cell types may be used to preferentially target specific types of neurons. Hemodynamic and ligand vectoring are complementary therapies. Ligand vectors may be pre-programmed to statically treat specific topographical areas of brainwave activity imbalance identified, for example, by a neuroimaging assessment. Hemodynamic vectors can dynamically treat in real-time systemic electrophysiological activity imbalances stimulated, for example, by psychotherapy, virtual reality, and/or perceptual events, as well as statically targeting specific regions of the brain. However, ligand vectors can transport biologics only to neurons that express the receptor types of interest by binding target receptors, which limits the types of neurons that can be treated with ligand vectors. Method 300 enables biologics to edit any type of neuron by activating the neuron prior to intranasal delivery of the biologic, which enables the biologic to edit the types of neurons that ligand vectoring cannot edit.

Method 300 may include a specific-purpose neurological condition reliever that treats individual conditions (for example, inattention, stress, anxiety, or depression) by altering neuron protein expression to modify neuron electrodynamics. Method 300 relieves the conditions by intranasal delivery of biologics to the brain in combination with (1) actively-directed hemodynamic vectoring, (2) passive hemodynamic vectoring, and/or (3) ligand vectors.

Method 300 may include either or both of treatment 300 and enhancement 400 steps, for example, as described in U.S. Patent Application Publication No. 2020/0254103 A1, which is incorporated by reference in its entirety. Treatment 300 generally includes treating a specific mental health condition and enhancement 400 generally includes general-purpose cognitive enhancements. Treatment 300 and enhancement 400 may be used together or separately as determined by a medical professional. Treatment 300 and enhancement 400 may be used to treat a variety of neurological medical conditions. Specifically, the method, including treatment 300 and enhancement 400 steps may be used to treat a wide variety of mental health conditions. In some specific instances, the method, treatment 300, and/or enhancement 400 steps may be used to treat ADD, Attention Deficit Hyperactivity Disorder (ADHD), PTSD, Obsessive Compulsive Disorder (OCD), stress disorders, anxiety, depression, sleep conditions, memory conditions, personality disorders, schizophrenia, concussions, head injuries, Tourette Syndrome, psychosomatic issues, tinnitus, conscious awareness issues, inattention, mind wandering, cravings, mental focus issues, concentration issues, mindfulness issues, meditation issues, and intuition issues.

Treatment steps 300 may include treatment steps 301-317 that enable a medical professional to treat a mental illness such as ADD/ADHD, PTSD, OCD, stress disorders, anxiety, depression, sleep conditions, memory conditions, concussions, head injuries, Tourette Syndrome, psychosomatic issues, and/or tinnitus. Before treatment 300 begins, a medical professional may prepare a patient with pre-treatment therapy. During pre-treatment therapy, the medical professional may set expectations that foster positive beliefs about change by employing psychological counseling techniques such as cognitive behavioral therapy, mindfulness-based stress reduction, neuro-linguistic programming, meditation, and/or visualization.

Treatment phase 300 may include step 301: a professional assessment to determine a condition to be treated. During step 301, a medical professional may assess the patient to identify a specific neurological issue, condition, and/or disorder to be treated. The medical professional can be a human or, in some instances, a computer program such as an artificial intelligence computer program.

Treatment phase 300 may also include step 302: identification of brain regions affected by the identified neurological issue, condition, and/or disorder to be treated. Neurological conditions and disorders may cause under- or over-activation in multiple areas in the brain. The medical professional may order a neuroimaging study, such as, without limitation, an fMRI, PET, or SPECT brain scan, to pinpoint area(s) in the patient's brain that may be over- or under-activated. The brain scan may show abnormal activity in one or more regions depending on the condition to be treated. Brain regions that are frequently affected by twelve common neurological conditions are shown in Table 1 below.

Treatment phase 300 may further include step 303: selecting a brain region to be treated to improve the condition. The medical professional may establish treatment priorities according to the degree of over- or under-activation of each region as revealed in the neuroimaging study, in some instances beginning with the most over- or under-activated area.

Treatment phase 300 may further include step 304: selecting an appropriate brain region activator to activate the target brain regions. The brain region activator activates the selected brain region to be treated to enable biologics to target the selected brain region. Specifically, the brain region activator is selected to activate a specific region of the brain such that biologics are absorbed into the selected brain region. Table 1 illustrates six examples of brain region activators that may be suitable for any one or more of at least twelve neurological treatment applications. Other brain region activation methods can be devised and employed by those skilled in the art. The activator examples shown are: transcranial pulsed ultrasound (TPU), transcranial magnetic stimulation (TMS), neurofeedback, perceptual isolation, virtual reality (VR), and psychotherapy.

As discussed above, TPU may involve the use of low intensity, low frequency ultrasound to stimulate the patient's brain and may be effective in a variety of applications because TPU can be focused in almost any area of the brain with a precision down to a few square millimeters. Furthermore, TPU may in some instances also increase the permeability of the blood-brain barrier to allow more effective transport of biologics in the bloodstream to the target brain regions.

As discussed above, TMS may include a noninvasive form of brain stimulation, in which a changing magnetic field is used to cause electric current at a specific area of the brain through electromagnetic induction. TMS has been used, for example, to treat depression. The effective range of TMS is maximal for neurons near the brain's surface, for example, in the cortex, with progressively less effect deep to the cortex.

Neurofeedback is a technique involving the use of a type of biofeedback that presents real-time feedback from brain activity to a patient/subject to reinforce healthy brain function through operant conditioning, and neurofeedback is effective across a wide spectrum of applications. Neurofeedback can be used to selectively activate almost any area in the brain of a human subject.

As defined above, perceptual isolation includes the deliberate reduction or removal of stimuli from one or more of the senses of a subject and may be used in applications addressing the default mode network (DMN) (posterior cingulate cortex, medial prefrontal cortex, angular gyrus) to quiesce neural activity in other areas of the brain and concentrate neural activity in the DMN.

VR may include simulating in whole or in part an experience that can be similar to or completely different from the real world. In some instances, VR has been used in applications treating aversive memories. A VR simulation can be used to strongly reactivate the memory, and methods of the invention can be used, for example, to attenuate the neuronal activity associated with the reactivated, unwanted memories.

Psychotherapy may include the use of psychological methods, particularly when based on regular personal interaction, to help a person change behavior, increase happiness, and/or overcome problems and can have efficacy in applications treating aversive memories, stress, anxiety, and/or depression. As discussed above, by reactivating unwanted memories or conditions, psychotherapy can be used with the methods of this disclosure to induce genetic or transcriptional therapies to attenuate their associated neuronal activity.

Treatment phase 300 may also include step 305: selecting a type of editing. According to methods provided herein, a medical professional may select a type of editing for treatment of the identified condition. For example, the medical professional may select one of at least transcriptional editing and genetic editing. Transcriptional editing may be used to produce a reversible, temporary result for testing purposes. Genetic editing is selected when the patient is ready for a permanent treatment.

Treatment phase 300 may further include step 306: selecting a neuron-editing biologic. In some instances, the brain region selected for treatment may be over- or under-active. If the region is underactive, then the medical professional may select biologics that raise neuron excitability to increase activity in the region. If the region is overactive, then the medical professional may select biologics that reduce neuron excitability to lower neuronal circuitry in the region. In some methods, a neuron's excitability can be reduced by decreasing its receptor population. One method to decrease the receptor population is to edit gene HTR2A or its transcriptional or translational pathways to reduce HTR2A's expression to lower the population of 5-hydroxytryptamine 2A receptors as described, for example, in U.S. Patent Application Publication No. 2019/0336585 A1, which is incorporated by reference in its entirety. If ligand vectoring is to be used, then the medical professional selects biologics with ligand vectors targeting the brain area to be edited.

Treatment phase 300 may also include step 307: calculating a dosage of biologics for the selected brain region. In this step, the medical professional may calculate an appropriate dose based on a plurality of factors including, but not limited to, the age, weight, gender, physical condition of the patient, the condition to be treated, and/or the region of the patient's brain to be targeted. When a biologic will be administered intranasally, then a dose may be selected that inversely correlates with performance on an olfactory test; performance on an olfactory test correlates with intranasal accessibility of the brain, and thus, lower doses of a biologic are necessary when a subject displays better performance on an olfactory test. An example of a dosing formula is included in U.S. Patent Application Publication No. 2019/0336585 A1, which is incorporated by reference in its entirety. If ligand vectoring is used in the method, then steps 308 and 309 of the treatment phase may be omitted, and a practitioner may instead proceed to step 310 described below.

Treatment phase 300 may further include step 308: calibrating the brain region activator. The medical professional calibrates the brain region activator method chosen in Step 304 discussed above to address the individual patient's brain region targeted for treatment. For transcranial pulsed ultrasound or transcranial magnetic stimulation, for example, the medical professional focuses the device on the target area in the patient's brain. For neurofeedback, the medical professional selects a neurofeedback program designed to address the patient's target brain region specifically. For perceptual isolation, the medical professional may select the stimuli reduction method to be used. For virtual reality, the medical professional may choose a VR simulation designed to evoke a particular memory or emotion related to the patient's issue. For psychotherapy, the medical professional may select a session protocol designed to evoke the patient's unwanted memory or condition.

The treatment phase 300 may also include step 309: quiesce brain activity. In this step, the patient may be placed in a resting state free from distractions to minimize brain activity to minimize activity in and/or deactivate all brain regions except the default mode network.

Treatment phase 300 may further include step 310: administering the calculated dose of the biologics to the subject. In this step, the biologics may be administered intranasally to the patient with a swab, aerosol, nasal inhaler, or irrigation protocol.

If ligand vectors are being used, then steps 311-313 may be omitted, and a practitioner may instead proceed to step 314 described below.

Treatment phase 300 may also include step 311: administering the selected brain region activator to activate the target brain region(s). In this step, the medical professional administers the brain region activator method selected for the patient in Step 304 discussed in detail above.

Treatment phase 300 may further include step 312: concentrating neural activity in the target brain region using a brain region activator. The brain region activator is used by the practitioner to concentrate neuronal activity in the target brain region(s) of the patient/subject receiving treatment.

Treatment phase 300 may also include step 313: transporting the biologics to the active neurons within the target brain region using hemodynamics. As discussed above, blood and cerebrospinal fluid containing glucose, oxygen, and biologics is transported to active neurons in the targeted area of the patient's brain by hemodynamics principles.

Treatment phase 300 may further include step 314: editing the active neurons using the biologics. Biologics may here be used to transfect neurons in the target area of the patient's brain and edit the neurons' DNA or RNA to normalize activity in the region as determined by the medical professional in the previous steps.

Treatment phase 300 may also include step 315: reducing symptoms of the condition. Normalized brain region activity relieves the symptoms of the patient's neurological condition.

Treatment phase 300 may further include step 316: iterating steps 302-315 as needed. If multiple brain areas are indicated for treatment, then a medical professional may return to step 302 discussed above to address the next area of priority. A brain scan performed at this juncture may be used to verify/assess the potentially normalized brain activity in the region just treated.

Treatment phase 300 may also include step 317: conducting post-treatment therapy. Post-treatment therapy may be used to instill appropriate mind and life practices in the subject to reinforce the biologic's effects by fostering supportive behavioral and epigenetic factors. Post-treatment therapy can employ psychological counseling protocols such as cognitive behavioral therapy, mindfulness-based stress reduction, neuro-linguistic programming, meditation, visualization, and/or other techniques.

Table 1 illustrates practical applications of the treatment 300 for twelve examples of common neurological conditions:

TABLE 1
practical applications of the treatment phase 300 of the method in treating twelve
examples of common neurological conditions.
Treatment

| | | | Brain region activator | | | | | |
|---|---|---|---|---|---|---|---|---|
| | | | | Transcranial | | | | |
| | | | Transcranial | magnetic | | Perceptual | Virtual | |
| Issue | | Main Brain regions affected | ultrasound | stim. | Neurofeedback | isolation | reality | Psychotherapy |
| 1. | ADD / ADHD | Amygdala, ventromedial prefrontal | • | | • | | | |
| | | cortex, hippocampus (under- | | | | | | |
| | | activated) | | | | | | |
| | | Dorsal attention and default mode | • | | • | • | | |
| | | networks (over-activated) | | | | | | |
| 2. | Traumatic | Amygdala, hippocampus | • | | • | | • | • |
| | memory / PTSD | Ventromedial prefrontal cortex | • | | • | | • | • |
| 3. | OCD | Orbital gyrus, caudate nucleus, | • | | • | | | |
| | | dorsal anterior cingulate cortex | | | | | | |
| 4. | Stress disorders | Amygdala, hypothalamus | • | | • | | | • |
| 5. | Anxiety | Amygdala | • | | • | | | • |
| 6. | Depression | Posterior cingulate cortex, | • | • | • | • | | |
| | | prefrontal cortex (underactive) | | | | | | |
| | | Amygdala, hippocampus, anterior | • | | • | | | |
| | | cingulate cortex (overactive) | | | | | | |
| 7. | Sleep issues | Prefrontal and parietal cortex, | • | | • | • | | |
| | | precuneus, anterior cingulate, | | | | | | |
| | | mesial temporal, thalamus and | | | | | | |
| | | hypothalamic arousal centers, | | | | | | |
| | | default-mode network | | | | | | |
| 8. | Memory | Hippocampus, amygdala | • | | • | | | |
| | concerns | | | | | | | |
| 9. | Concussions | Frontal and temporal lobes | • | | • | | | |
| 10. | Tourette | Basal ganglia | • | | | | | |
| | Syndrome | | | | | | | |
| 11. | Psychosomatic | Amygdala, hippocampus | • | | • | | | • |
| | issues | Ventromedial prefrontal cortex | • | | • | | | • |
| 12. | Tinnitus | Auditory cortex | • | | | | | |

In applying the methods of this disclosure, a medical practitioner should be aware of the characteristics of the neurological and/or psychiatric condition being treated and should devise specific treatment phase 300 methodologies as discussed with respect to the twelve examples provided in Table 1.

First, ADD and ADHD may involve altered activation of the amygdala, ventromedial prefrontal cortex, and/or hippocampus, depending on the ADD or ADHD subtype. Activity in these areas can be corrected by administering biologics designed to alter neuron excitability in conjunction with transcranial pulsed ultrasound or neurofeedback brain region activation techniques that direct the biologics to the amygdala, ventromedial prefrontal cortex, and/or hippocampus via hemodynamic vectoring.

ADD and ADHD may also involve overactivation of the dorsal attention and default mode networks. Activity in these areas can be reduced by administering biologics designed to alter neuron excitability in conjunction with transcranial pulsed ultrasound, neurofeedback, and/or perceptual isolation brain region activation techniques, which direct the biologics to the dorsal attention and/or default mode networks via hemodynamic vectoring.

In some subjects, PTSD may involve overactivation of the amygdala and/or hippocampus. These areas can be corrected by administering biologics for altering neuron excitability in conjunction with transcranial pulsed ultrasound, neurofeedback, virtual reality, and/or psychotherapy brain region activation techniques selected to direct the biologics to the amygdala and/or hippocampus via passive hemodynamic vectoring.

PTSD may also be characterized by altered activation of the ventromedial prefrontal cortex. Activity in this area can be corrected by biologics for changing neuron excitability in conjunction with transcranial pulsed ultrasound, neurofeedback, virtual reality, and/or psychotherapy brain region activation techniques that direct the biologics to the ventromedial prefrontal cortex via hemodynamic vectoring.

Neuroimaging studies of people with OCD reveal hyperactivity in the dorsal anterior cingulate cortex, orbital gyrus, and caudate nucleus. Activity in these areas can be normalized by administering biologics to reduce neuron excitability together with transcranial pulsed ultrasound or neurofeedback brain region activation techniques that direct the biologics to the dorsal anterior cingulate cortex, orbital gyrus, and/or caudate nucleus via hemodynamic vectoring.

Individuals with stress disorders exhibit hyperactivity in the amygdala and hypothalamus. These areas can be brought into balance by administering biologics for altering neuron excitability combined with transcranial pulsed ultrasound, neurofeedback, or psychotherapy brain region activation techniques that direct the biologics to the amygdala and/or hypothalamus via hemodynamic vectoring.

Individuals experiencing anxiety exhibit hyperactivity in the amygdala, which can be normalized by administering biologics for altering neuron excitability combined with transcranial pulsed ultrasound, neurofeedback, or psychotherapy brain region activation techniques that direct the biologics to the amygdala via hemodynamic vectoring.

Depression involves disrupted activation of the posterior cingulate cortex and prefrontal cortex. Activity in these areas can be corrected by administering biologics designed to normalize neuron excitability in conjunction with transcranial pulsed ultrasound, transcranial magnetic stimulation, neurofeedback, and/or perceptual isolation brain region activation techniques that direct the biologics to the posterior cingulate cortex and/or prefrontal cortex via hemodynamic vectoring.

Depression is also associated with altered activation of the amygdala, hippocampus, and anterior cingulate cortex. Activity in these areas can be normalized by administering biologics designed to change neuron excitability in conjunction with transcranial pulsed ultrasound or neurofeedback brain region activation techniques that direct the biologics to the amygdala, hippocampus, and/or anterior cingulate cortex via hemodynamic vectoring.

Insomnia and other sleep issues can involve altered activation of the prefrontal and parietal cortex, precuneus, anterior cingulate, mesial temporal, thalamus, hypothalamus, and the default-mode network. Activity in these areas can be normalized by administering biologics for correcting neuron excitability in conjunction with transcranial pulsed ultrasound, neurofeedback, and/or perceptual isolation brain region activation techniques that direct the biologics to the prefrontal and parietal cortex, precuneus, anterior cingulate, mesial temporal, thalamus, hypothalamus, and/or the default-mode network via hemodynamic vectoring.

Neuroimaging studies of individuals with memory problems reveal subnormal activity in the hippocampus and amygdala. These areas can be normalized by administering biologics for altering neuron excitability in conjunction with transcranial pulsed ultrasound or neurofeedback brain region activation techniques that direct the biologics to the hippocampus and/or amygdala via hemodynamic vectoring.

Head injuries may pathologically change neuronal activity in the frontal and temporal lobes. Corrected activity in these areas can be promoted by administering biologics for increasing neuron excitability together with transcranial pulsed ultrasound or neurofeedback brain region activation techniques that direct the biologics to the frontal and/or temporal lobes via hemodynamic vectoring.

Studies of individuals with Tourette Syndrome show abnormal activity in the basal ganglia. Activity can be normalized by administering biologics for increasing neuron excitability along with transcranial pulsed ultrasound brain region activation techniques that direct the biologics to the basal ganglia via hemodynamic vectoring.

Psychosomatic issues may involve overactivation of the amygdala and hippocampus. These areas can be normalized by administering biologics for changing neuron excitability in conjunction with transcranial pulsed ultrasound, neurofeedback, or psychotherapy brain region activation techniques that direct the biologics to the amygdala and/or hippocampus via hemodynamic vectoring.

Psychosomatic issues may also cause altered activation of the ventromedial prefrontal cortex. Activity in this area can be corrected by biologics for normalizing neuron excitability in conjunction with transcranial pulsed ultrasound, neurofeedback, or psychotherapy brain region activation techniques that direct the biologics to the ventromedial prefrontal cortex via hemodynamic vectoring.

Tinnitus/persistent ringing in the ears is associated with overactivated auditory connections. Activity can be reduced by administering biologics for decreasing neuron excitability along with transcranial pulsed ultrasound brain region activation techniques that direct the biologics to the auditory cortex via hemodynamic vectoring.

The enhancement phase 400 of the methods disclosed herein may include enhancement steps 401-417 that enable a medical professional to enhance a subject's cognition, including areas such as, without limitation, conscious awareness issues, inattention, mind wandering, cravings, mental focus issues, concentration issues, mindfulness issues, meditation issues, and/or intuition issues. As a typical initial step before the enhancement phase 400 is initiated, a medical professional prepares the patient with pre-treatment therapy. During this pre-treatment therapy, the medical professional may set expectations that foster positive beliefs about change by employing psychological counseling techniques such as cognitive behavioral therapy, mindfulness-based stress reduction, neuro-linguistic programming, meditation, and/or visualization.

Enhancement phase 400 may include step 401: professional psychological assessment to verify a patient's suitability for cognitive enhancement. General-purpose genetic cognitive enhancement is generally suitable for adults in sound mental and emotional health. As a result, during step 401, a medical professional may conduct a psychological assessment of the patient to screen out candidates who do not meet these criteria, such as, for example, individuals with alcohol or substance abuse, bipolar disorder, depression, schizophrenia, or other psychological conditions or disorders. The assessment also ensures the candidate is not currently taking any drugs, medications, or substances that could interfere with the normal, natural functioning of their brain such as, for example, certain prescription drugs, alcohol, caffeine, nicotine, cannabis, nootropics, ginseng, or other similar substances or herbal preparations. In some instances, the medical professional who conducts this step can be a human or, in some instances, a computer program such as an artificial intelligence computer program.

Enhancement phase 400 may also include step 402: conduct a psychological assessment to determine the patient's cognitive goals. The medical professional may conduct a psychological assessment to ascertain the patient's cognitive enhancement goals. This assessment may cover several topics including the type of cognitive enhancement the patient desires and whether the cognitive upgrade is to be permanent or temporary.

Enhancement phase 400 may further include step 403: selecting a brain region to be treated to achieve the cognitive goals previously identified. Target brain regions for thirteen specific cognitive enhancement applications are illustrated in Table 2. Genetic or transcriptional engineering can be used to optimize neuron performance in these regions to achieve the patient's goals. Depending on the application, optimization may involve increasing or decreasing neuronal activity. If ligand vectors are being used, step 404 may be omitted, and a medical professional may instead proceed to step 405 described below.

Enhancement phase 400 may further include step 404: selecting an appropriate brain region activator to activate the target brain regions. The brain region activator activates the selected brain region to be treated to enable the biologics to target the selected brain region. Specifically, the brain region activator is specifically selected to activate a specific region of the brain such that the biologics are directed to and absorbed into the selected brain region. Table 2 illustrates six examples of brain region activators suitable for thirteen exemplary cognitive enhancement applications. Other brain region activation methods can be devised by those skilled in the art based on the teachings provided herein. The activator examples shown are identical to the ones described in Step 304 described above.

Enhancement phase 400 may also include step 405: selecting a type of editing. The medical professional may select a type of editing for treatment of the identified condition. For example, the medical professional may select one of at least transcriptional editing and genetic editing. Transcriptional editing may be used to produce a reversible, temporary result in a patient, such as for testing purposes, allowing the patient and/or practitioner to evaluate the effects of the enhancement on a temporary basis. Genetic editing may be selected when the patient is ready for a permanent treatment.

Enhancement phase 400 may further include step 406: selecting a neuron-editing biologic. The brain region selected for enhancement may be over- or under-active. If the region is underactive, then the medical professional may select biologics that raise or lower neuron excitability to normalize activity in the region. If the region is overactive, then the medical professional may select biologics that reduce neuron excitability to normalize the region. For example, a neuron's excitability can be reduced by decreasing its receptor population, which can increase its electrical resistance. One way decreasing the receptor population is to edit gene HTR2A or its transcriptional or translational pathways to reduce HTR2A's expression, which will lower the population of 5-hydroxytryptamine 2A receptors as described, for example, in U.S. Patent Application Publication No. 2019/0336585 A1, which is incorporated by reference in its entirety. If ligand vectoring is used, then the medical professional may select biologics with ligand vectors targeting the brain area to be edited.

Enhancement phase 400 may also include step 407: calculating a dose for the selected brain region. The medical professional calculates an appropriate dose for the biologics based on a plurality of factors including, but not limited to, the age, weight, gender, and/or physical condition of the patient, the condition to be treated, and/or the region of the patient's brain to be targeted. When a biologic will be administered intranasally, then a dose may be selected that inversely correlates with performance on an olfactory test; performance on an olfactory test correlates with intranasal accessibility of the brain, and thus, lower doses of a biologic are necessary when a subject displays better performance on an olfactory test. An example of a dosing formula is included in U.S. Patent Application Publication No. 2019/0336585 A1, which is incorporated herein by reference. If ligand vectoring is used, then steps 408 and 409 may be skipped.

Enhancement phase 400 may further include step 408: calibrating the brain region system specific connectome. The medical professional calibrates the brain region method selected in Step 404 to address the patient's targeted brain region. For transcranial pulsed ultrasound or transcranial magnetic stimulation, the medical professional may focus the device on a target area in the patient's brain. For neurofeedback, the medical professional selects a neurofeedback program designed to address the patient's target brain region. For perceptual isolation, the medical professional selects the stimuli reduction method to be used. For virtual reality, the medical professional chooses a VR simulation designed to evoke a particular memory or emotion related to the patient's therapeutic goals. For psychotherapy, the medical professional selects a session protocol that evokes the patient's unwanted memory or condition.

Enhancement phase 400 may also include step 409: quiesce brain activity. The patient may be placed in a resting state free from distractions to minimize brain activity in an effort to deactivate all brain regions except the default mode network of the patient.

Enhancement phase 400 may further include step 410: administering the calculated dose of the biologics. The biologics are administered intranasally to the patient with a swab, aerosol, nasal inhaler, or irrigation protocol. If ligand vectors are used, then a medical practitioner may skip steps 411-413 and instead proceed to step 414 as described below.

Enhancement phase 400 may also include step 411: administering the brain region activator to activate the target brain regions. The medical professional administers the brain region activator method selected for the patient in Step 404.

Enhancement phase 400 may further include step 412: concentrating neural activity in the target brain region using the brain region connectome method. The brain region connectome method concentrates neuronal activity in the target brain region(s) as discussed above.

Enhancement phase 400 may also include step 413: transporting the biologics to active neurons within the target brain region using hemodynamics. Cerebral blood flow containing glucose, oxygen, and biologics is transported to active neurons in the targeted area of the patient's brain, which facilitates administration of the selected biologic to the appropriate area of the brain.

Enhancement phase 400 may further include step 414: editing the active neurons using the biologics. Biologics transfect neurons in the target area of the patient's brain and edit the neurons' DNA or RNA to enhance activity in the region.

Enhancement phase 400 may also include step 415: enhancing the patient's cognitive capacity. Optimized brain region activity may operate to expand the patient's cognitive capacity as desired.

Enhancement phase 400 may further includes step 416: iterating steps 402-415 as needed. If multiple brain areas are indicated for treatment, then the medical professional returns to Step 402 to address the next area of priority. A brain scan performed at this juncture verifies optimized brain activity in the region just treated.

Enhancement phase 400 may also include step 417: conducting post-treatment therapy. Post-treatment therapy may be used to teach/instill mind and life practices for reinforcing the biologic's effects into the subject by fostering supportive behavioral and epigenetic factors. This therapy can employ psychological counseling protocols such as cognitive behavioral therapy, mindfulness-based stress reduction, neuro-linguistic programming, meditation, visualization, and other techniques.

Table 2 illustrates the practical application of the enhancement 400 in treating thirteen examples of common cognitive enhancements:

TABLE 2
practical application of the enhancement 400 in treating thirteen examples of common cognitive enhancements
Enhancement

| | | | Transcranial | | | | |
|---|---|---|---|---|---|---|---|
| | | Transcranial | magnetic | | Perceptual | Virtual | |
| Objective | Brain Regions | ultrasound | stim. | Neurofeedback | isolation | reality | Psychotherapy |

| | | | | | | |
|---|---|---|---|---|---|---|
| 1. | Raise conscious awareness | Posterior | • | • | | |
| 2. | Decrease inattention | cingulate cortex | • | • | | |
| 3. | Reduce mind-wandering | (ventral) | • | • | | |
| 4. | Lower craving | | • | • | | |
| 5. | Sharpen mental focus | | • | • | | |
| 6. | Increase concentration | | • | • | | |
| 7. | Enhance mindfulness | | • | • | | |
| 8. | Improve meditation | | • | • | | |
| 9. | Increase intuition | | • | • | | |
| 10. | Improve ESP | Caudate | • | | | |
| 11. | Cognitive therapy for | Various | | | | • |
| | unwanted behaviors | | | | | |
| 12. | Cognitive therapy for | Various | | | • | • |
| | desired behaviors | | | | | |
| 13. | Alleviate cognitive | Posterior | • | • | | |
| | impairment symptoms | cingulate cortex | | | | |

Table 2 lists several exemplary practical applications for achieving cognitive enhancement. Several of these, such as objectives 1-9, include general-purpose cognitive enhancements: raising conscious awareness, decreasing inattention, lowering craving, sharpening mental focus, increasing concentration, and improving meditation. These cognitive enhancements 1-9 may be achieved by administering biologics to decrease neuron excitability along with transcranial pulsed ultrasound and/or perceptual isolation brain region activation techniques that direct the biologics to the ventral posterior cingulate cortex via hemodynamic vectoring. Reduced posterior cingulate cortex activity correlates with increased attention, conscious awareness, mental acuity, clarity, focus, concentration, and mindfulness, as well as reduced mind-wandering, inattention, and cravings.

Regarding cognitive enhancement 10 listed in Table 2, studies have shown that individuals can demonstrate extra-sensory perception (ESP). In this disclosure, ESP refers to any perception other than as mediated by vision, hearing, olfaction, taste, and touch. Patients who desire to raise their ESP abilities may benefit from administering biologics for increasing neuron excitability along with transcranial pulsed ultrasound brain region activation techniques that direct the biologics to specific brain regions via hemodynamic vectoring.

Regarding cognitive enhancement 11 listed in Table 2, psychotherapy session protocols can be designed to stimulate the underlying causes of the patient's unwanted behavior, including attitudes, emotions, beliefs, expectations, and memories. This mental activity will generate brainwave and neural activity in corresponding areas in the patient's brain. Biologics administered for reducing neuron excitability may then be transported to these active areas via hemodynamic vectoring.

Regarding cognitive enhancement 12 listed in Table 2, psychotherapy session protocols and immersive multi-sensory VR programs can be designed to help the patient visualize and experience desired behaviors, including attitudes, feelings, beliefs, expectations, and assumptions. This mental activity will generate neural activity in corresponding areas in the patient's brain. Biologics administered for altering neuron excitability can be transported to these active areas via hemodynamic vectoring.

Regarding cognitive enhancement 13 listed in Table 2, mild cognitive impairment may affect many different regions of the brain. It can be treated indirectly by administering a cognitive enhancement protocol to offset its effects. This protocol involves administering biologics for decreasing neuron excitability in the posterior cingulate cortex in conjunction with transcranial pulsed ultrasound or perceptual isolation techniques that direct the biologics to the posterior cingulate cortex via hemodynamic vectoring.

Various aspects of this disclosure relate to a method to treat symptoms of anxiety or depression in a subject, comprising providing an AAV vector and administering the AAV vector to the subject, wherein the AAV vector is an AAV9 vector; the AAV vector comprises a nucleic acid that comprises a nucleotide sequence that encodes an interfering RNA; the interfering RNA comprises a reverse complement sequence that is a reverse complement of an RNA transcript that encodes human 5HT-2A receptor; the interfering RNA has at least 95 percent sequence identity with at least 20 consecutive nucleotides set forth in SEQ ID NO. 4; the nucleotide sequence encodes a promoter that is operably linked to the sequence encoding the interfering RNA such that the promoter can mediate transcription of the interfering RNA in neurons; the promoter is a human MECP2 promoter; the AAV vector is administered intranasally; at least 200 billion particles of the AAV vector are administered to the subject; the AAV vector is administered in an amount sufficient to achieve an MOI per target cell of at least 3; the target cells are neurons of the brain; the administering transfects the neurons with the AAV9 vector; transfection of the neurons with the AAV9 vector causes expression of the interfering RNA; expression of the interfering RNA reduces the concentration of the RNA transcript in the neurons; the reduction of the concentration of the RNA transcript in the neurons reduces expression of the human 5HT-2A receptor in the brain of the subject by at least 20 percent; and reduced expression of the human 5HT-2A receptor in the brain of the subject treats the symptoms of anxiety or depression.

Various aspects of this disclosure relate to a method to modulate brain activity in a brain of a subject, comprising providing an AAV vector and administering the AAV vector to the brain of the subject, wherein: the AAV vector comprises a nucleic acid that comprises a nucleotide sequence that encodes an interfering RNA; the interfering RNA comprises a reverse complement sequence that is a reverse complement of an RNA transcript that encodes a human neurotransmitter receptor; the administering transfects neurons of the brain with the AAV vector; transfection of the neurons with the AAV vector causes expression of the interfering RNA; expression of the interfering RNA reduces the concentration of the RNA transcript in the neurons; the reduction of the concentration of the RNA transcript in the neurons reduces expression of the human neurotransmitter receptor in the brain; and reduced expression of the human neurotransmitter receptor in the brain of the subject modulates the brain activity. In some embodiments, the interfering RNA is a shRNA.

In some embodiments, the AAV vector is an AAV9 vector.

In some embodiments, the human neurotransmitter receptor is a G protein-coupled receptor.

In some embodiments, the human neurotransmitter receptor is a G protein-coupled receptor, and binding of a neurotransmitter to the human neurotransmitter receptor activates phospholipase C to cause calcium release. In some specific embodiments, the human neurotransmitter receptor is a G protein-coupled receptor; binding of a neurotransmitter to the human neurotransmitter receptor activates phospholipase C to cause calcium release; and the administering modulates brain activity in the subject by modulating neurotransmitter-dependent calcium release in neurons that are transfected with the AAV vector.

In some embodiments, the human neurotransmitter receptor is 5-hydroxytryptamine receptor 2A (5HT-2A receptor).

In some embodiments, the human neurotransmitter receptor is a G protein-coupled receptor, and binding of a neurotransmitter to the human neurotransmitter receptor inhibits adenylyl cyclase to decrease cAMP. In some specific embodiments, the human neurotransmitter receptor is a G protein-coupled receptor; binding of a neurotransmitter to the human neurotransmitter receptor inhibits adenylyl cyclase to decrease cAMP; and the administering modulates brain activity in the subject by modulating cAMP concentration in neurons that are transfected with the AAV vector.

In some embodiments, the human neurotransmitter receptor is 5-hydroxytryptamine receptor 1A (5HT-1A receptor).

In some embodiments, the interfering RNA comprises a nucleotide sequence selected from SEQ ID NO. 5, 7, 8, 9, 10, and 11.

In some embodiments, the interfering RNA has at least 95 percent sequence identity with at least 20 consecutive nucleotides set forth in SEQ ID NO. 4. In some specific embodiments, the interfering RNA has at least 95 percent sequence identity with at least 40 consecutive nucleotides set forth in SEQ ID NO. 4. In some even more specific embodiments, the interfering RNA has at least 95 percent sequence identity with at least 60 consecutive nucleotides set forth in SEQ ID NO. 4. In some very specific embodiments, the interfering RNA has at least 95 percent sequence identity with SEQ ID NO. 4.

In some embodiments, the interfering RNA comprises a nucleotide sequence that includes at least 20 consecutive nucleotides set forth in SEQ ID NO. 4. In some specific embodiments, the interfering RNA comprises a nucleotide sequence that includes at least 40 consecutive nucleotides set forth in SEQ ID NO. 4. In some even more specific embodiments, the interfering RNA comprises a nucleotide sequence that includes at least 60 consecutive nucleotides set forth in SEQ ID NO. 4. In some very specific embodiments, the interfering RNA comprises a nucleotide sequence that includes SEQ ID NO. 4.

In some embodiments, the nucleotide sequence encodes a promoter that is operably linked to the sequence encoding the interfering RNA such that the promoter can mediate transcription of the interfering RNA in neurons, wherein the promoter is a human MECP2 promoter or a human alpha-synuclein promoter. In some embodiments, the promoter is a human MECP2 promoter.

In some embodiments, the method comprises administering the AAV vector in an amount sufficient to achieve a MOI per neuron of at least 1, wherein the AAV vector is administered intranasally. In some specific embodiments, the method comprises administering the AAV vector in an amount sufficient to achieve a MOI per neuron of at least 3, wherein the AAV vector is administered intranasally. In some very specific embodiments, the method comprises administering the AAV vector in an amount sufficient to achieve a MOI per neuron of at least 5, wherein the AAV vector is administered intranasally.

In some embodiments, the AAV vector is administered intranasally.

In some embodiments, the method comprises administering an olfactory test to the subject prior to administering the AAV vector, wherein the AAV vector is administered intranasally. In some specific embodiments, the method comprises administering an olfactory test to the subject prior to administering the AAV vector and adjusting the number of particles of the AAV vector that are administered based on the results of the olfactory test, wherein the AAV vector is administered intranasally; more particles of the AAV vector are administered if the olfactory test indicates that the cribriform plate and adjacent olfactory epithelium of the subject are less permeable to transit of biologics from the intranasal cavity into the olfactory bulb and adjacent regions of the forebrain; and less particles of the AAV vector are administered if the olfactory test indicates that the cribriform plate and adjacent olfactory epithelium of the subject are more permeable to transit of biologics from the intranasal cavity into the olfactory bulb and adjacent regions of the forebrain. In some very specific embodiments, the method comprises administering an olfactory test to the subject prior to administering the AAV vector and adjusting the number of particles of the AAV vector that are administered based on the results of the olfactory test, wherein the AAV vector is administered intranasally; more particles of the AAV vector are administered if the olfactory test indicates that the subject has a poor sense of smell because a poor sense of smell indicates that the cribriform plate and adjacent olfactory epithelium of the subject are less permeable to transit of biologics from the intranasal cavity into the olfactory bulb and adjacent regions of the forebrain; and less particles of the AAV vector are administered if the olfactory test indicates that the subject has a normal sense of smell because a normal sense of smell indicates that the cribriform plate and adjacent olfactory epithelium of the subject are more permeable to transit of biologics from the intranasal cavity into the olfactory bulb and adjacent regions of the forebrain.

In some embodiments, the method comprises focusing pulsed ultrasound in regions of the brain, wherein the regions of the brain in which the pulsed ultrasound is focused display reduced expression of the human neurotransmitter receptor per neuron relative to other regions of the brain in which the pulsed ultrasound is not focused. Transcranial pulsed ultrasound can be configured to activate neurons in target regions of the brain, which activity increases blood flow to the target regions, which increases the flow of therapeutics (such as the AAV vector) to the target regions.

In some embodiments, the method comprises focusing pulsed ultrasound in regions of the brain, wherein the regions of the brain in which the pulsed ultrasound is focused display reduced expression of the human neurotransmitter receptor per neuron relative to other regions of the brain in which the pulsed ultrasound is not focused.

In some embodiments, the method comprises focusing repetitive Transcranial Magnetic Stimulation in regions of the brain, wherein the regions of the brain in which the repetitive Transcranial Magnetic Stimulation is focused display reduced expression of the human neurotransmitter receptor per neuron relative to other regions of the brain in which the repetitive Transcranial Magnetic Stimulation is not focused. Transcranial Magnetic Stimulation can be configured to activate neurons in target regions of the brain, which activity increases blood flow to the target regions, which increases the flow of therapeutics (such as the AAV vector) to the target regions.

In some embodiments, the region comprises at least a portion of one or more of the amygdala, dorsal amygdala, hippocampus, hypothalamus, interpeduncular nucleus, anterior cingulate cortex, dorsal anterior cingulate cortex, posterior cingulate cortex, prefrontal cortex, ventromedial prefrontal cortex, pons, pontine nuclei, cerebellum, Purkinje layer of the cerebellum, orbital gyrus, and caudate nucleus. In some specific embodiments, the region comprises one or more of the olfactory bulb, cortex, subcortical regions, cerebellum, Purkinje cell layer of the cerebellum, pons, pontine gray nuclei, interpeduncular nucleus, thalamus, hypothalamus, hippocampus, and amygdala. In some very specific embodiments, the region comprises one or more of the olfactory bulb, cortex, cerebellum, Purkinje cell layer of the cerebellum, pons, pontine gray nuclei, and interpeduncular nucleus.

In some embodiments, the method comprises readministering the AAV vector a period of time following the administering. In some specific embodiments, the method comprises readministering the AAV vector to the subject at least 2 months and up to 24 months following the administering. In some very specific embodiments, the method comprises readministering the AAV vector to the subject at least 6 months and up to 12 months following the administering. Mice that received an AAV vector encoding an shRNA displayed therapeutic effects eight weeks following administration of the AAV vector, which suggests that humans who receive the AAV vector encoding an shRNA will display therapeutic effects for at least eight weeks. The inventors contemplate that the duration of therapeutic effects of such AAV vectors in humans will be significantly more persistent than the therapeutic effects observed in mice such that frequency of dosing will be temporally dilated in humans relative to mice. Additionally, neurons do not undergo mitosis that might otherwise dilute episomal copies of DNA encoding an interfering RNA, and which therefore allows persistent, durable therapeutic effects.

Various aspects of this disclosure relate to a method to treat a mental health condition in a subject, comprising determining that the subject presents with the mental health condition and performing a method as described anywhere in this disclosure such as a method to modulate brain activity in a brain of a subject.

In some embodiments, the mental health condition is stress, anxiety, depression, ADD, OCD, PTSD, bipolar disorder, a personality disorder, memory concern, dementia, or inattention. In some specific embodiments, the mental health condition is anxiety, or a symptom of the mental health condition is anxiety. In some very specific embodiments, the mental health condition is anxiety.

Various aspects of this disclosure relate to a method to reduce body weight in a subject, comprising performing a method as described anywhere in this disclosure such as a method to modulate brain activity in a brain of a subject. In some embodiments, the method is effective at reducing body fat in the subject.

Various aspects of this disclosure relate to a method to treat a mental health condition in a subject, comprising determining that the subject presents with the mental health condition, and performing the method as described elsewhere in this disclosure such as a method to modulate brain activity in a brain of a subject, wherein the mental health condition is stress, anxiety, depression, ADD, OCD, PTSD, bipolar disorder, a personality disorder, memory concern, dementia, or inattention. In some embodiments, the mental health condition is anxiety or depression, or a symptom of the mental health condition is anxiety or depression. In some specific embodiments, (1) the mental health condition is anxiety or depression, or a symptom of the mental health condition is anxiety or depression, and (2) the method is effective to treat anxiety or depression.

Various aspects of this disclosure relate to a recombinant nucleic acid that encodes an interfering RNA that has at least 95 percent sequence identity with at least 20 consecutive nucleotides set forth in SEQ ID NO. 4. In some embodiments, the recombinant nucleic acid encodes an interfering RNA that has at least 95 percent sequence identity with at least 40 consecutive nucleotides set forth in SEQ ID NO. 4. In some specific embodiments, the recombinant nucleic acid encodes an interfering RNA that has at least 95 percent sequence identity with at least 60 consecutive nucleotides set forth in SEQ ID NO. 4. In some very specific embodiments, the recombinant nucleic acid encodes an interfering RNA that has at least 95 percent sequence identity with SEQ ID NO. 4.

In some embodiments, the recombinant nucleic acid encodes an interfering RNA that includes at least 20 consecutive nucleotides set forth in SEQ ID NO. 4. In some specific embodiments, the recombinant nucleic acid encodes an interfering RNA that includes at least 40 consecutive nucleotides set forth in SEQ ID NO. 4. In some even more specific embodiments, the recombinant nucleic acid encodes an interfering RNA that includes at least 60 consecutive nucleotides set forth in SEQ ID NO. 4. In some very specific embodiments, the recombinant nucleic acid encodes an interfering RNA that comprises a nucleotide sequence that includes SEQ ID NO. 4.

Various aspects of this disclosure relate to a recombinant nucleic acid that encodes an interfering RNA that comprises a nucleotide sequence selected from SEQ ID NO. 5, 7, 8, 9, 10, and 11.

In some embodiments, the recombinant nucleic acid is RNA. In some specific embodiments, the nucleic acid is an interfering RNA. In some very specific embodiments, the nucleic acid is an shRNA.

In some embodiments, the nucleic acid is an shRNA; the shRNA comprises a first sequence, a second sequence, and a third sequence; the first sequence and the third sequence form intramolecular base pair interactions that hybridize the first sequence to the third sequence; the second sequence is juxtaposed between the first sequence and the third sequence; and the second sequence comprises a hairpin turn that lacks intramolecular base pair interactions. The second sequence may comprise, for example, up to 13 consecutive nucleotides of the nucleotide sequence set forth in SEQ ID NO. 11. The first sequence may be SEQ ID NO. 5, for example, and the third sequence may be SEQ ID NO. 7, 8, or 10.

In some embodiments, the recombinant nucleic acid is single-stranded DNA. The AAV9 vectors of this disclosure, for example, comprise recombinant nucleic acids that are single-stranded DNA.

In some embodiments, the recombinant nucleic acid comprises a promoter that is operably linked to a sequence encoding the interfering RNA such that the promoter can mediate transcription of the interfering RNA in neurons. In some specific embodiments, the promoter is a human MECP2 promoter or an alpha-synuclein promoter. In some very specific embodiments, the promoter is human MECP2 promoter.

Various aspects of this disclosure relate to an AAV vector comprising a recombinant nucleic acid as described anywhere in this disclosure. In some embodiments, the AAV vector is an AAV9 vector.

In some embodiments, the recombinant nucleic acid comprises ITRs. In some specific embodiments, the recombinant nucleic acid comprises two ITRs that are configured to package the recombinant nucleic acid in an AAV vector.

In some embodiments, the recombinant nucleic acid is double-stranded DNA.

In some embodiments, the recombinant nucleic acid is a plasmid. In some specific embodiments, the recombinant nucleic acid is a plasmid that comprises an origin of replication. In some even specific embodiments, the recombinant nucleic acid is a plasmid that comprises a bacterial origin of replication. In some very specific embodiments, the recombinant nucleic acid is a plasmid that comprises a bacterial origin of replication and an f1 origin of replication.

In some embodiments, the recombinant nucleic acid comprises an antibiotic resistance gene. In some specific embodiments, the recombinant nucleic acid comprises an ampicillin resistance gene.

Various aspects of this disclosure relate to an AAV vector comprising a recombinant nucleic acid as described herein. In some embodiments, the AAV vector is an AAV9 vector.

Various aspects of this disclosure relate to a cell line comprising a recombinant nucleic acid as described herein. In some embodiments, the cell line is a human cell line. In some specific embodiments, the cell line comprises HEK293 cells.

Various aspects of this disclosure relate to a composition comprising a AAV vector as described herein and cesium chloride.

### Example 1: Design of Guide RNA to Knockout HTR2A in Mouse Neurons

The HTR2A mouse gene encodes a single protein-coding transcript HTR2A-201. Two in silico strategies were designed to knockout the gene with both strategies utilizing the same cut site for Cas9 in the first coding exon. In the first strategy, a Cas9-directed double-strand break near the start site (ATG) would be repaired by non-homologous end joining (NHEJ), introducing random INDELs and potential frameshift mutations. In the second strategy, CRISPR/Cas9 would cut the first coding exon, and homology-directed repair (HDR) systems would lead to insertion of a STOP-pA cassette to terminate transcription of the HTR2A gene. The targeting sequence chosen in both cases, TGCAATTAGGTGACGACTCGAGG (SEQ ID NO: 1), would give no predicted off-target cut sites, produce an 86.6 percent frameshift frequency, and have a precision score of 0.55. An additional 200 base pair (bp) knock-in STOP-pA donor cassette was cloned that would insert two stop codons, which are underlined below:

(SEQ ID NO: 2)
GAAGACAATATCTCCCTGAGCTCAATTCCAAACTCCTTAATGCAATTAGG
TGACTAGTGAATTCAACTTGTTTATTGCAGCTTATAATGGTTACAAATAA
AGCAATAGCATCACAAATTTCACAAATAAAGCATTTTTTTCACTGCTCGA
GGCTCTACCCTAATGACTTCAACTCCAGGGATGCTAACACTTCCGAAGCC

The 200 base pair knock-in STOP-pA donor cassette (SS ODN template) requires HDR, a process that mainly occurs in the S and G2 phases of the cell cycle and is thought to rarely take place in mature neurons. If HDR-mediated insertion of the SS ODN sequence does not occur, it is still possible to produce potential genomic changes in postmitotic neurons by random INDELs induced by Cas9 cleavage and non-homologous end-joining (NHEJ) repair introducing premature stop codons. Thus, the strategy ensured potential gene editing within neurons regardless of whether HDR or NHEJ were the predominant repair systems.

### Example 2. Design of Adeno-Associated Viral Vector Platform

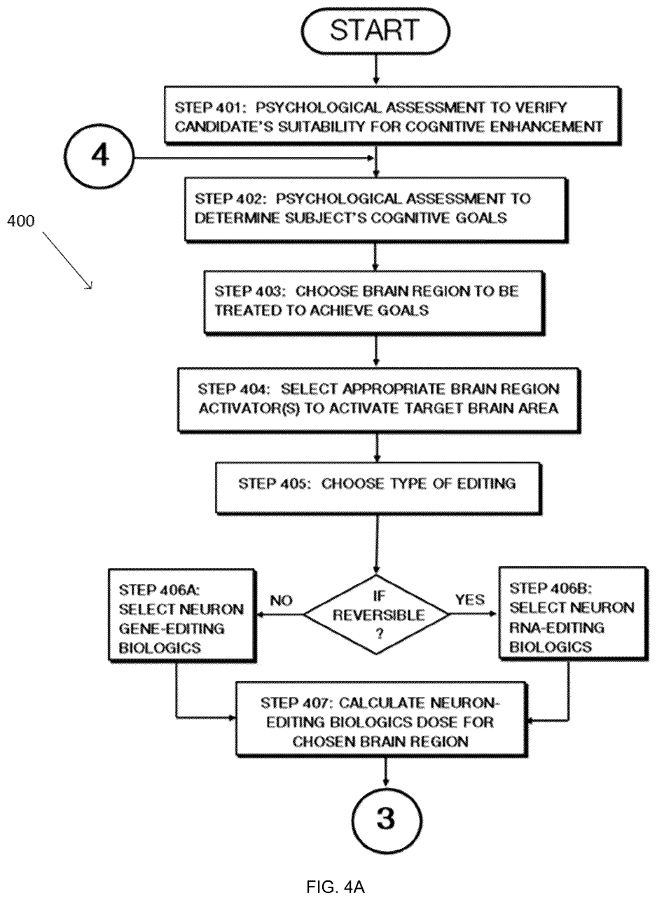

Two different adeno-associated virus serotype 9 (AAV9) vectors were designed to deliver spCas9 and gRNA to CNS neurons. The design of two vectors was necessary based on the limited carrying capacity of 4.7 kb for AA9 viruses. Dual AAV9 systems were created to expand the capacity. The first AAV9 expressed spCas9 under a neuronal-specific promoter, MECP2 (~4.2 kb), and the spCas9 vector utilized the PX551 plasmid from Addgene (pAAV9-Mecp2-spCas9; FIG. 5). The second AAV9 vector consisted of the gRNA sequence, 200-bp SS ODN donor template, and a green-fluorescence protein (GFP) reporter under the U6 promoter (AAV9-gRNA-U6-GFP). Large-scale transfection of plasmids was carried out in HEK293 cells, and viral vectors were purified through a series of cesium chloride centrifugations. Titer load (in genome copy number per ml, or GC/ml) was determined by quantitative real-time PCR, with typical yields of 20 trillion GC/ml. Both AAV9 vectors were stored in PBS with 5 percent glycerol at −80 °C until used.

### Example 3. Administration of AAV Vectors to Mice

4-5 weeks old male CD-1 mice were group-housed at 5 or 6 mice per cage in a temperature-controlled room (21-22 °C) with a light-dark cycle (12 h/12h; lights on: 17:30-05:30; lights off: 05:30-17:30) and with food and water available ad libitum. Mice were acclimated to the animal facility for at least 1-week before beginning experimental procedures.

The AAV-treated group received an equal mixture of AAV9-gRNA-U6-GFP and AAV9-Mecp2-spCas9 suspended in 0.9 percent NaCl (saline). The concentration of AAV9 stock mixture was ~5 trillion GC/ml. For each behavioral test described below, a new, independent batch of AAV9 vectors was prepared. The AAV cocktail was administered twice on day one (morning and afternoon) five weeks before the behavioral tests. For intranasal delivery of AVV, mice were hand-restrained with the nose positioned to facilitate the dosing. A meniscus of AAV solution droplet (10 microliters per nostril) was then formed at the tip of the micropipette and presented for inhalation in each of the nares of the mouse. Each mouse received a total of 40 microliters of AAVs equivalent to ~200 billion viral particles. Vehicle-treated animals received 40 microliters of saline for each treatment following the same protocol.

### Example 4. Verification of Knockout by qPCR

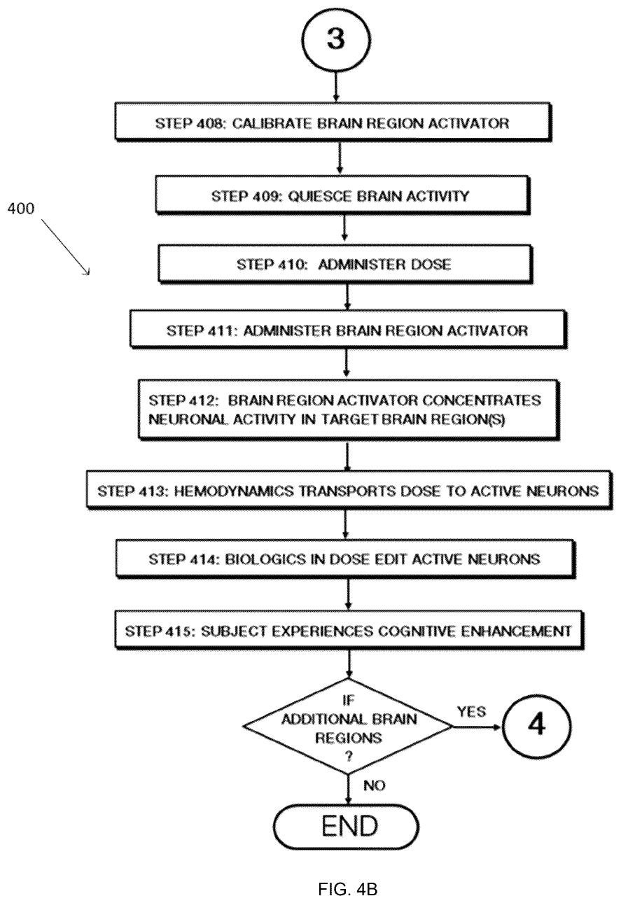

Following treatment of mice on day one, mice were sacrificed five weeks post intranasal treatment, and brains were snap-frozen. Total RNA was extracted from vehicle control (N=5) and AAV-treated mice (N=5) using a standard extraction protocol with TRIzol™ and then dissolved in diethyl pyrocarbonate-treated deionized water and quantified. Following reverse transcription, qPCR was performed. Treatment led to a significant 8.47-fold decrease in HTR2A expression (p=0.05) (FIG. 6).

To analyze gene editing, whole brain (N=5) or olfactory bulb (N=5) genomic DNA was analyzed by next-generation sequencing around the predicted Cas9 cleavage site of the HTR2A gene. An INDEL frequency of 0.07-0.13 percent was detected in all 5 brain samples. In all examined brains, the INDEL profile revealed single base pair deletions of adenine at identical positions near the PAM site. Other INDELs observed were single base deletions in cytosine at the same position in 3 out of 4 brains and in guanine in one brain sample. A similar result was obtained in the olfactory bulb samples from the same five animals.

For the single base pair deletions in adenine, these two INDELs would result in a predicted frame-shift mutation leading to the insertion of premature stop codons. None of the INDELs resulted from an insertion of the 200 base pair donor SS ODN repair template, which suggests that NHEJ is the primary method of repair for generation of nonsense mutations. The low frequency of editing is unsurprising given the large dilution effect of brain genomic DNA. Neurons make up less than 10 percent of the total number of cells in the brain, and only a subpopulation of these neurons is targeted by the AAV cargo. Taken together, these results support CRISPR-mediated gene editing of HTR2A in vivo that is most likely occurring through the single-nucleotide INDEL formation and generation of premature stop codons via NHEJ.

### Example 5. Verification of mRNA Knockout by Microscopy

Five weeks following intranasal AAV9 delivery of co-packaged Cas9 DNA and a HTR2A-targeting gRNA, immunofluorescence studies were conducted in fixed, sagittal mouse sections to examine 5HT-2A receptor protein expression. Mice were anesthetized with a 5 percent isoflurane/oxygen mixture and sacrificed by decapitation. Brains with olfactory bulbs were extracted and fixed in 4 percent formalin for 48 hours and transferred to vials containing 1 percent formalin in PBS buffer.

gRNA expression was visualized by GFP fluorescence while 5HT-2A receptor labeling was accomplished utilizing an antibody previously demonstrated to be highly specific for mouse 5HT-2A receptor. Following dehydration, 4 micrometer paraffin-embedded, sagittal sections were cut lateral to the midline and used for immunofluorescence labeling. Briefly, all tissue sections were labeled with anti-GFP antibody (rabbit mAB #2956) 1:1,000 (Cell Signaling Technology, Inc., Danvers, MA, USA) or anti-5HT-2A receptor antibody (rabbit polyclonal, #24288) at 1:500 dilution (Immunostar, Hudson, WI, USA). Secondary antibodies were conjugated to FITC or Cy3. DAPI was used as a nuclear stain. Whole slide scanning was performed using a Pannoramic Midi II scanner using a 40× objective lens with optical magnification of 98× at 0.1 micrometer per pixel.

5HT-2A receptor fluorescence was quantified using ImageJ software. This was accomplished by capturing 50× immunofluorescence images from three different fields (cortex, cerebellum, and subcortical regions) in three separate tissue sections from each group (vehicle control or AAV-treated). All data were expressed as the mean gray value±SEM. The mean gray value reflects the sum of the gray values of all the pixels in the selected area divided by the number of pixels. The area of selection in square pixels was not significantly different between vehicle-controls (676,181) and AAV-treated (671,647), p=0.639, N=9 for each group.

Intranasal administration of the AAV9 vectors (final concentration of mixture was ~200 billion viral particles) resulted in widespread expression of GFP/gRNA throughout the CNS including olfactory bulb, cortex, and sub-cortical areas including the interpeduncular nucleus (IPN) (FIG. 7), a major connectome for stress-mediated pathways. In AAV-treated mice, GFP (gRNA) labeling was strongest in the cell bodies of neurons, and, when colocalized with 5HT-2A in AAV-treated mice, staining of 5HT-2A appeared weaker in the cell bodies with some labeling still apparent within apical dendrites. As expected, little evidence of GFP (gRNA) staining was observed in vehicle-control mice.

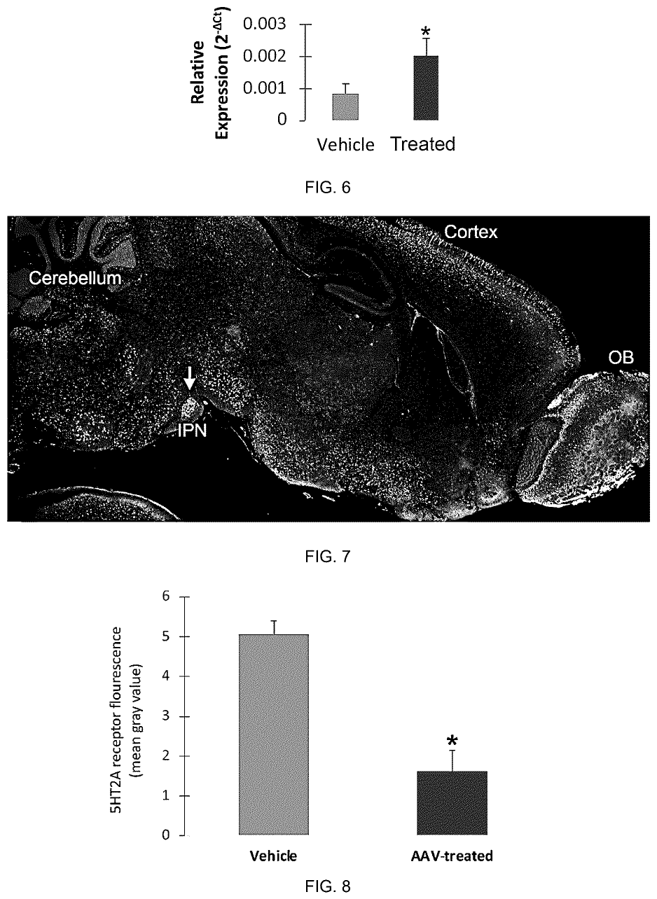

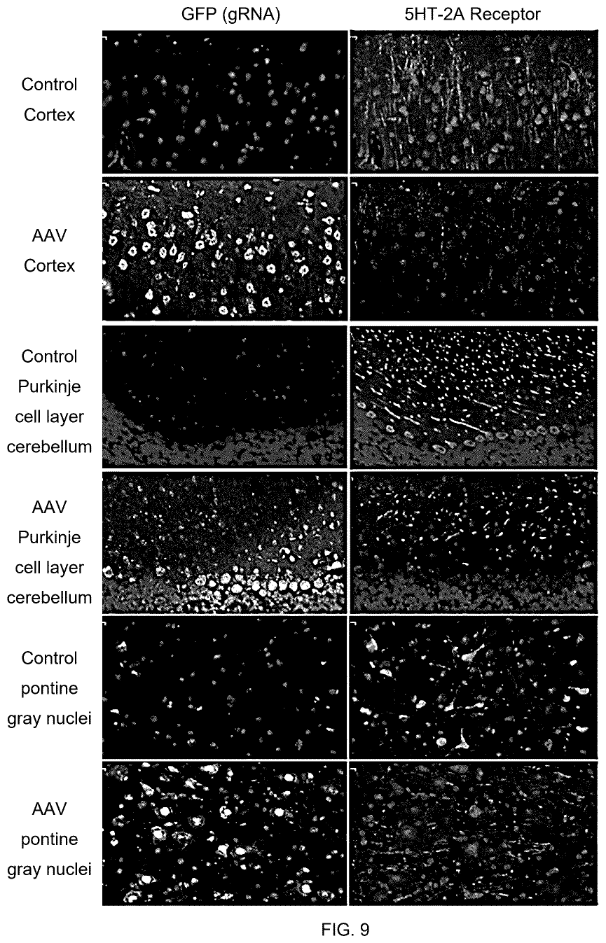

ImageJ quantification indicated a significant 68 percent decrease in 5HT-2A receptor fluorescence of AAV-treated mice as compared to vehicle controls (FIG. 8), p=0.0026. These results suggest that treatment of the co-packaged spCas9 DNA and a HTR2A-targeting gRNA led to a decrease in 5HT-2A receptor staining. Co-localization of gRNA/GFP together with 5HT-2A receptor fluorescence was evident throughout the CNS including layers I-III of the cortex, the Purkinje cell layer of the cerebellum, and subcortical areas including within pontine gray nuclei (FIG. 9). While strong 5HT-2A receptor labeling was evident in vehicle controls in these regions, staining appeared to be less robust in AAV-treated mice. Importantly, knockout of the 5HT-2A occurred in brain regions linked to anxiety-mediated pathways including the interpeduncular nucleus and pontine gray nuclei.

### Example 6. Marble Burying Test

The marble burying test is used to record the number of marbles buried by mice placed in a novel environment. The theoretical basis for marble burying as an anxiety-related behavior stems from findings that anxiolytics and SSRIs reduce this behavior and from the well-documented innate response of mice to bury threating objects.

The marble-burying test apparatus consisted of transparent polycarbonate cages (30 cm×18 cm×19 cm) containing a 5 cm layer of fine sawdust bedding and 20 glass marbles (diameter: 1.5 cm) spaced evenly along the walls of the cage. Each animal was placed individually in the cage where it remained for a 20-minute test session. On termination of the test session, the animals were removed from the cage, and the number of marbles with at least two-thirds of the marbles buried in the sawdust was counted. Anxious mice bury more marbles.

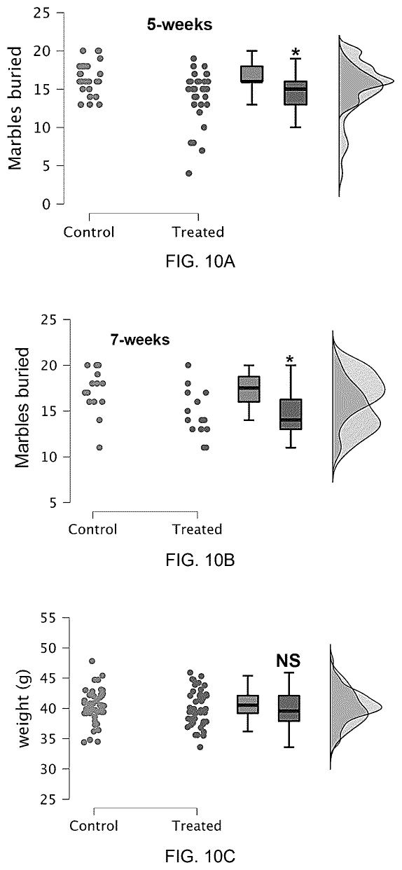

Results for 5-week AAV-treated mice indicated a 14.8 percent decrease in the number of marbles buried compared to vehicle-controls (N=30, p=0.0008) (FIG. 10A). Similar results were obtained following a doubling of the dose at 7-weeks post treatment (N=15, p=0.013) (FIG. 10B). Following treatment of mice with co-packaged Cas9 DNA and gRNA on day 1, there were no reported observational differences in mouse behavior, and treated mice did not differ significantly in weight at the end of the 5-week trial (p=0.569), (FIG. 10C) suggesting that no major off-target edits in critical genes occurred.

### Example 7. Light-Dark Behavioral Test

The light/dark box test is a well-characterized method to evaluate the relative anxiety status of mice. The light/dark paradigm in mice is based on a conflict between the innate aversion to brightly illuminated areas and spontaneous exploratory activity. If given a choice, mice prefer the dark box. Anxiolytic compounds increase the total duration of time spent in the lit area as well as the number of entries into the lit box area.

Mice were given a choice between exploring a brightly lit chamber or a dark chamber; anxious mice will spend more time in the dark chamber. The light/dark box apparatus consisted of two PVC (polyvinylchloride) boxes (19×19×15 cm) covered with plexiglass. One of these boxes was opaque and not illuminated. The other box was illuminated by a desk lamp placed above and providing approximately 2000 Lux. An opaque plastic tunnel (5×7×10 cm) separated the dark box from the illuminated one. A camera linked to a video tracking system (Viewpoint, France) was used to monitor the behavior of the mouse in the lit box. Animals were placed individually in the lit box with their heads directed toward the tunnel. Time spent in the lit box and the number of transitions between the two boxes was recorded over a 5-minute period after the first entry of the animal in the dark box. The total distance traveled in the lit box was also recorded.

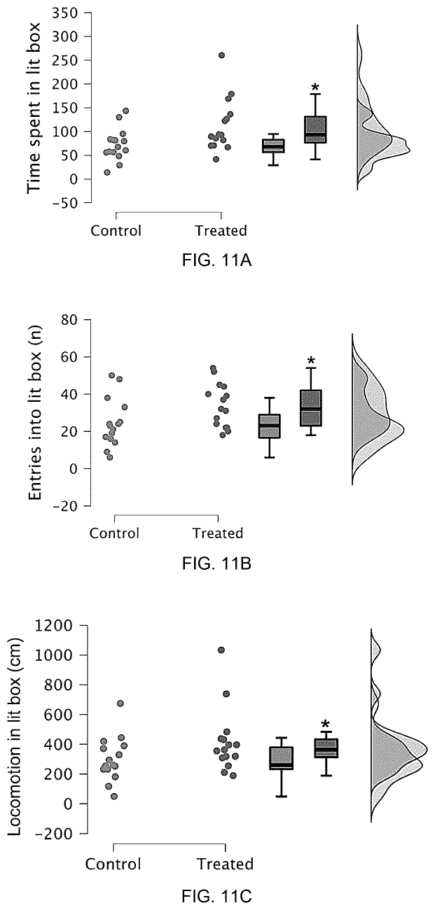

AAV-treated mice spent increased time in the lit box compared to vehicle-controls (35.7% increase, p=0.024), greater numbers of entries into the lit box (27.5% increase, p=0.024), and greater total locomotion within the lit box (27.8%, p=0.049) (FIG. 11A-11C). A regression analysis between total 5HT-2A receptor protein fluorescence and time spent in the lit box indicated a moderate negative linear correlation (r=−0.55). For context, these results were compared with the acute effects of diazepam (1 mg/kg po, 1 hour). AAV-treatment led to comparative results to diazepam in the time spent in the lit box (35.7% vs 40%) (FIG. 11D), with diazepam outperforming AAV-treated mice in the number of entries into the lit box (27.5% vs 55%) (FIG. 11E). Thus, intranasal adeno-associated virus delivery of co-packaged Cas9 DNA and a HTR2A-targeting gRNA significantly decreased anxiety and performed on par with the benzodiazepine diazepam.

### Example 8. Elevated Plus Maze Test

The ability of the designed CRISPR/Cas9 cargo to decrease anxiety was also assessed with an EPM test. The EPM test is commonly used to evaluate anxiety in rodents. Male C57Bl/6J mice from Jackson Labs were utilized. Mice were received at 6-7 weeks of age and allowed to acclimate for 1-2 weeks prior to testing at an average age of 8 weeks. All animals were examined, handled, and weighed prior to initiation of the study to assure adequate health and suitability. During the course of the study, 12/12 light/dark cycles was maintained. Food and water were provided ad libitum for the duration of the study. Each mouse was randomly assigned across the treatment groups. The test was performed during the animal's light cycle phase.

The EPM has two opposite open arms and two closed arms. Mice will sometimes explore and spend time in the open arms, but anxious mice tend to first enter and spend most of the test time in the closed arms. The maze consists of two closed arms (14.5 h×5 w×351 cm) and two open arms (6 w×351 cm) forming a cross, with a square center platform (6×6 cm). All visible surfaces were black acrylic. Each arm of the maze was placed on a support column 56 cm above the floor. Antistatic black vinyl curtains (7′ tall) surround the EPM to make a 5′×5″ enclosure. Animals were brought into the experimental room at least 1 hour before the test to acclimate. Mice were placed in the center of the EPM facing the closed arm for a 5-min run. All animals were tested once. The number of entries in the closed and open arms were automatically recorded by a computer, and the EPM was thoroughly cleaned after each mouse. Testing was conducted at week 5 of intranasal dosing as described above with each mouse (n=15) receiving a total of 40 microliters of AAVs equivalent to ~200 billion viral particles. Vehicle-treated animals (n=15) received 40 microliters of saline for each treatment following the same protocol.

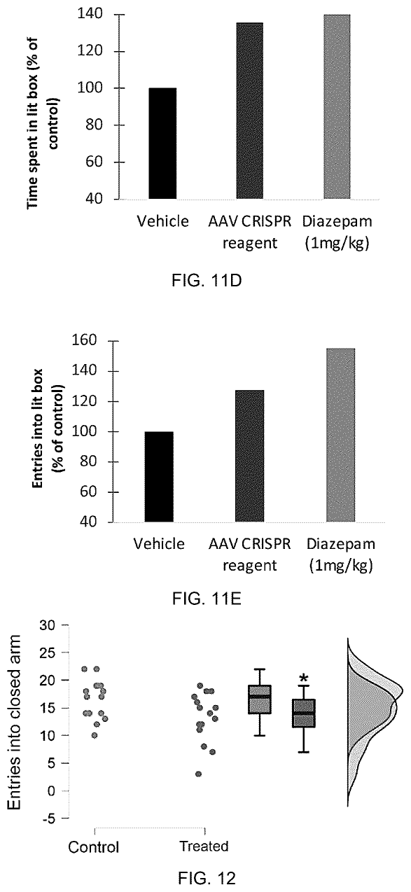

At 5 weeks post-dosing, mice exhibited a significant 24 percent decrease in the number of entries into the closed arm (FIG. 12) (p=0.017). In addition, the AAV-treated cohort resulted in less distance traveled into closed arms (p=0.032). There was no significant difference between the two groups in the number of entries into the open arm (p=0.50). A documented 17 percent increase in the time spent in open arms was observed, however, this increase did not reach statistical significance due to the large variance observed in the AAV-treated group (p=0.257). There were no significant differences in weight between the two groups (p=0.183).

### Example 9: Design of Interfering RNA to Knock Down HTR2A Expression in Mouse Neurons and Light-Dark Behavioral Tests

An in silico strategy was designed to knockdown HTR2A expression in mouse neurons using interfering RNA. The strategy utilized an AAV vector to deliver a nucleotide sequence encoding an interfering RNA that is a reverse complement of mouse HTR2A mRNA such that co-expression of the interfering RNA and HTR2A mRNA in a mouse neuron results in (1) annealing of the interfering RNA to the HTR2A mRNA to form double-stranded RNA and (2) Dicer-mediated hydrolysis of the double-stranded RNA. The nucleotide sequence also included inverted terminal repeats (ITRs) for packaging single-stranded DNA into an AAV vector as well as a mouse MECP2 promoter for expression of the interfering RNA in neurons. The mouse interfering RNA had the sequence

(SEQ ID NO. 3)
GCUGAGCACAUCCAGGUAAAUCCAGGUUUUGGCCACUGACUGACCUGGAU
UUCUGGAUGUGCUCAG

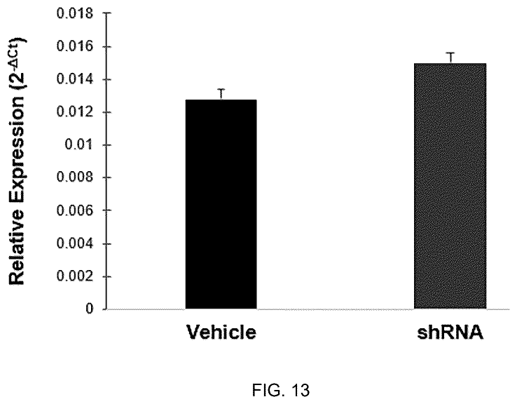

An AAV9 vector was prepared to carry the single-stranded DNA encoding the interfering RNA using a strategy similar to the strategy described in Example 2. The AAV9 vector was administered to mice using a method similar to the method described in Example 3. The HTR2A mRNA knock down was confirmed by reverse transcription followed by qPCR, which identified a reduction in HTR2A mRNA, for example, in mouse olfactory bulbs for knock down mice relative to vehicle-treated controls. The reduction in HTR2A mRNA displayed statistical significance at p-value=0.006 441 (FIG. 13).

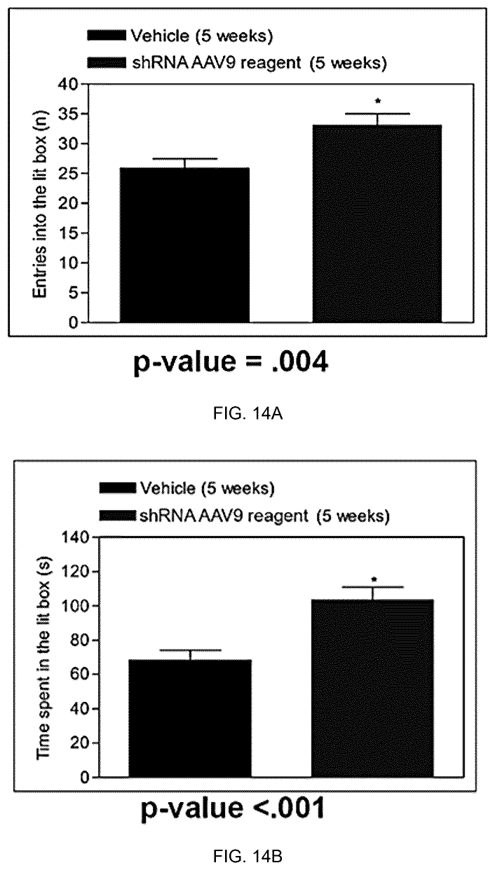

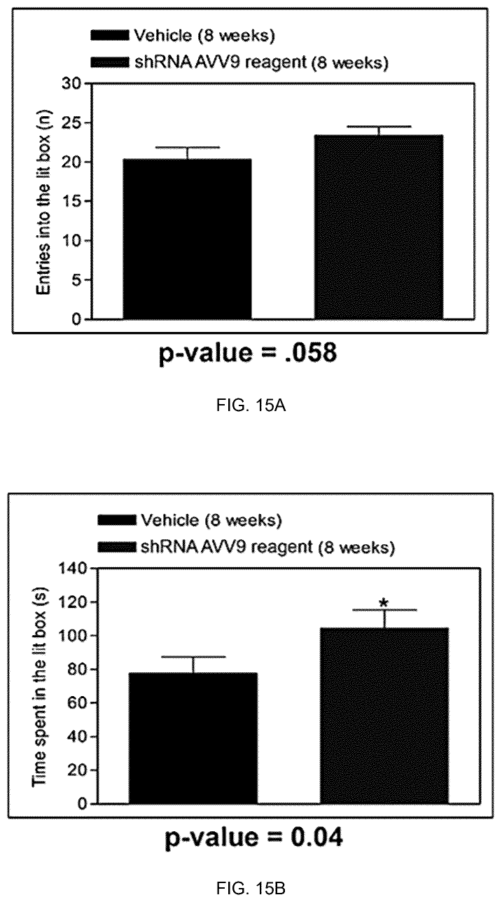

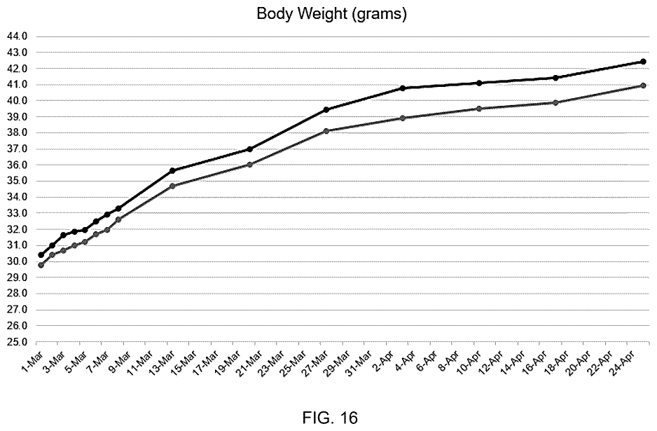

The behavior of treated mice and vehicle controls was assessed with a light/dark box test similar to the test described in Example 7. At 5-weeks following treatment, a statistically-significant increase in entries into the lit box was observed for knockdown mice treated with the AAV vector relative to vehicle controls (FIG. 14A), and a statistically-significant increase in time spent in the lit box was observed for knockdown mice relative to vehicle controls (FIG. 14B). At 8-weeks following treatment, an increase in entries into the lit box was observed for knockdown mice treated with the AAV vector relative to vehicle controls (FIG. 15A), and a statistically-significant increase in time spent in the lit box was observed for knockdown mice relative to vehicle controls (FIG. 15B). The increase in entries into the lit box was not statistically significant at 8-weeks, but the p-value for AAV9-treated mice versus vehicle controls at 8-weeks was nevertheless 0.058, which suggests that the observed difference was real and that the research was simply insufficiently powered to observe statistical significance at p-value<0.5.

The foregoing research suggests that an interfering RNA strategy utilizing intranasal administration of an AAV vector can effectively knock down HTR2A expression sufficiently enough to cause behavioral changes in mice that are consistent with an anxiolytic effect.

### Example 10: Design of Interfering RNA to Knock Down HTR2A Expression in Human Neurons

An in silico strategy was designed to knockdown HTR2A expression in human neurons using interfering RNA. The strategy utilized a AAV vector to deliver a nucleotide sequence encoding an interfering RNA that is a reverse complement of human HTR2A mRNA such that co-expression of the interfering RNA and HTR2A mRNA in a neuron results in (1) annealing of the interfering RNA to the HTR2A mRNA to form double-stranded RNA and (2) Dicer-mediated hydrolysis of the double-stranded RNA. The nucleotide sequence also included inverted terminal repeats (ITRs) for packaging single-stranded DNA into an AAV vector and a human MECP2 promoter for expression of the interfering RNA in neurons.

The nucleotide sequence of the interfering RNA is GCUGUUCUGAAGACAAAGAACUCUGGUUUUGGCCACUGACUGACCAGAGUUC UGUCUUCAGAACAG (SEQ ID NO. 4). This nucleotide sequence includes the sequence UUCUGAAGACAAAGAACUCUG (SEQ ID NO. 5), which is the reverse complement of human HTR2A mRNA sequence CAGAGUUCUUUGUCUUCAGAA (SEQ ID NO. 6). The interfering RNA sequence SEQ ID NO. 4 also includes the sequences CAGAGUUCU (SEQ ID NO. 7) and UGUCUUCAGAA (SEQ ID NO. 8), which are subsequences of human HTR2A mRNA sequence SEQ ID NO. 6, and which allows the interfering RNA to form hairpin structures by intramolecular base pairing. The subsequence CUGUUCUGAAGACAAAGAACUCUGGU (SEQ ID NO. 9) of SEQ ID NO. 4 contains SEQ ID NO: 5 and is capable of base pairing with the subsequence ACCAGAGUUCUGUCUUCAGAACAG (SEQ ID NO. 10) of SEQ ID NO. 4, which contains SEQ ID NO. 7 & 8. SEQ ID NO. 10 contains a deletion of two consecutive uridine phosphate nucleotides relative to three consecutive adenosine phosphate nucleotides in SEQ UD NO. 9, which lowers the affinity of SEQ ID NO. 10 for SEQ ID NO. 9 relative to the affinity of human HTR2A mRNA sequence SEQ ID NO. 6 for SEQ ID NO. 9. Human HTR2A mRNA can therefore displace SEQ ID NO. 10 from SEQ ID NO. 9 to form an intermolecular complex between the interfering RNA and human HTR2A mRNA, which targets the human HTR2A mRNA for Dicer-mediated cleavage and thereby knocks down human HTR2A expression. The deletion of two uridine phosphate nucleotides in SEQ ID NO. 10 corresponds to a deletion of two of three consecutive uridine phosphate nucleotides that are underlined in SEQ ID NO. 6 above such that SEQ ID NO. 10 only contains one uridine phosphate nucleotide at this position, which uridine phosphate nucleotide is underlined in SEQ ID NO. 10 above. The single uridine phosphate results in a gap in the interfering RNA when SEQ ID NO. 10 anneals to SEQ ID NO. 9, which gap occurs at three consecutive adenosine phosphate nucleotides that are underlined in SEQ ID NO. 9 above because the three consecutive adenosine phosphate nucleotides lack the corresponding two of three consecutive uridine phosphate nucleotides in SEQ ID NO. 10 to form three intramolecular base pairs. This gap results in the reduced affinity of SEQ ID NO. 10 for SEQ ID NO. 9 relative to the affinity of human HTR2A mRNA sequence SEQ ID NO. 6 for SEQ ID NO. 9. Finally, human interfering RNA sequence SEQ ID NO. 4 has the same loop region GUUUUGGCCACUGACUGAC (SEQ ID NO. 11) as mouse interfering RNA sequence SEQ ID NO. 3.

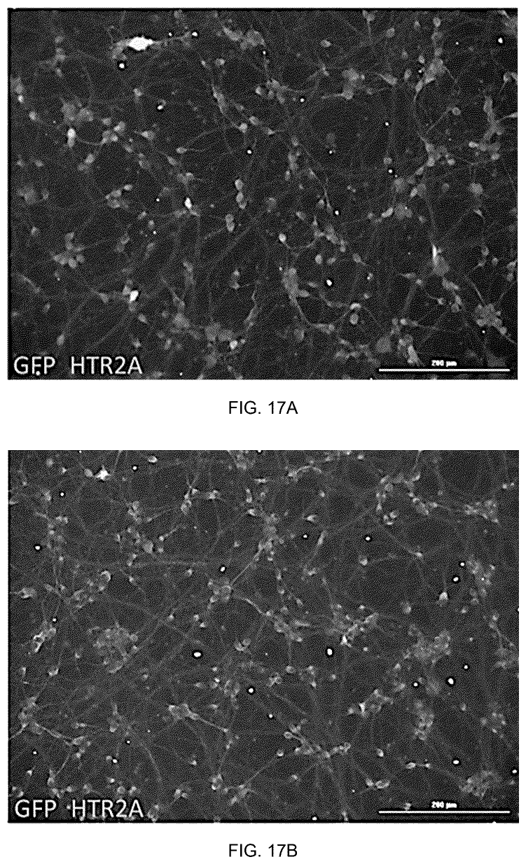

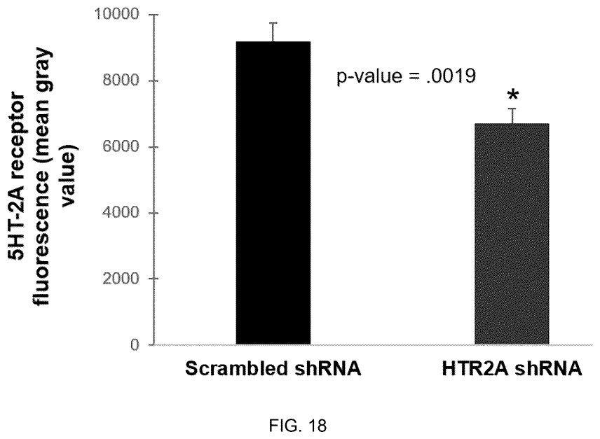

An AAV9 vector was prepared to carry the single-stranded DNA encoding the interfering RNA using a strategy similar to the strategy described in Example 2. A control AAV9 vector encoding a scrambled version of the interfering RNA was also prepared. The interfering RNA and control vectors were used to transfect human glutamatergic neurons in a 96-well plate seeded at 30,000 neurons per well with a multiplicity of infection of about 300,000. The neurons were immunostained for 5HT-2A receptor (FIGS. 17A and 17B). The neurons that were treated with the interfering RNA AAV9 vector displayed a statistically significant 26.8 percent decrease in 5HT-2A receptor expression relative to neurons treated with the AAV9 vector that encoded the scrambled interfering RNA (p-value=0.0019). These results suggest that AAV9 vectors encoding the interfering RNA according to SEQ ID NO. 4 can effectively knock down HTR2A mRNA in human neurons.

### Example 11: Plasmids for Propagating Nucleic Acids Encoding Interfering RNAs for Incorporation into AAV Vectors

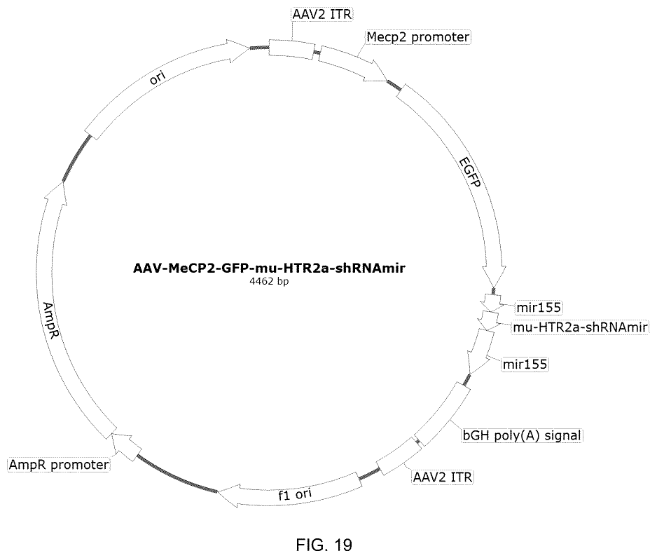

Plasmid AAV-MeCP2-GFP-mu-HTR2a-shRNAmir is depicted in FIG. 19, and its sequence is set forth in SEQ ID NO. 12. This plasmid is used to propagate a nucleic acid for incorporation into an AAV9 vector to knock down HTR2A expression in mice as described in Example 9.

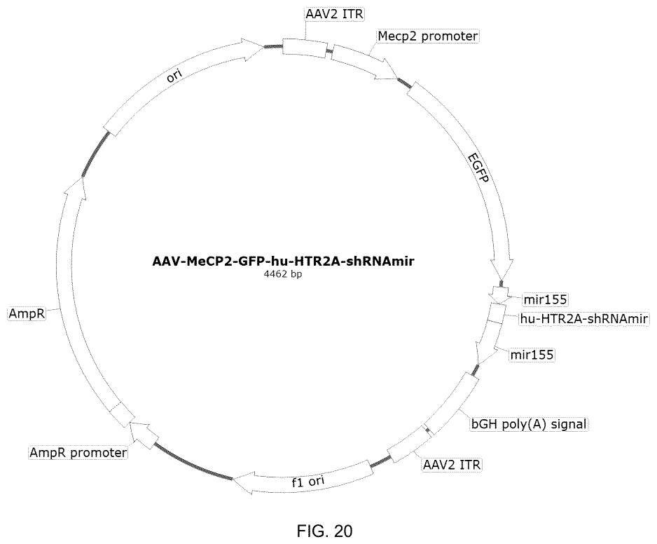

Plasmid AAV-MeCP2-GFP-hu-HTR2A-shRNAmir is depicted in FIG. 20, and its sequence is set forth in SEQ ID NO. 13. This plasmid is used to propagate a nucleic acid for incorporation into an AAV9 vector to knock down HTR2A expression in humans as described in Example 10.

Each plasmid includes two inverted terminal repeats (AAV2 ITR), which allow the packaging of DNA between the AAV2 ITR into AAV vectors. The AAV2 ITRs are from AAV serotype 2, which are compatible with AAV9. Each plasmid depicts a MECP2 promoter, which drives transcription of a GFP (EGFP; enhanced green fluorescent protein), and which drives transcription of an interfering RNA (mu-HTR2a-shRNAmir or hu-HTR2A-shRNAmir). The skilled person will immediately recognize that GFP expression will be disabled for clinical trials and therapeutic use in humans, for example, by excising the entire GFP nucleotide sequence from the plasmid vectors. The interfering RNA is a short hairpin RNA for both plasmids. The interfering RNA was designed based murine miR-155 (mir155) and utilizes a terminal loop (SEQ ID NO. 11) from murine miR-155 as well as 5′ and 3′ sequences from mirR-155. Each transcript ends with a poly-adenosine tail from bovine growth hormone (bGH poly(A) signal). Each plasmid also includes an origin of replication for propagation in bacteria (ori) and an fl origin of replication (fl ori) for generating single-stranded DNA for packaging into an AAV vector. Each plasmid also includes an ampicillin resistance gene (AmpR) for selection of bacteria that contain the plasmid.

The foregoing description, for purpose of explanation, has been described with reference to specific embodiments. However, the illustrative disclosure above is not exhaustive and does not limit the inventions to the precise forms disclosed. Many modifications and variations are possible in view of the foregoing teachings. The embodiments were chosen and described to best explain the principles of the present systems and methods and their practical applications to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

1. A method to treat symptoms of anxiety or depression in a subject, comprising:
providing an adeno-associated virus (AAV) vector; and
administering the AAV vector to the subject,
wherein:
the AAV vector is an AAV serotype 9 (AAV9) vector;
the AAV vector comprises a nucleic acid that comprises a nucleotide sequence that encodes an interfering RNA;
the interfering RNA comprises a reverse complement sequence that is a reverse complement of an RNA transcript that encodes human 5HT-2A receptor;
the interfering RNA has at least 95 percent sequence identity with at least 20 consecutive nucleotides set forth in
(SEQ ID NO. 4)
GCUGUUCUGAAGACAAAGAACUCUGGUUUUGGCCACUGACUGACCAGAGU
UCUGUCUUCAGAACAG;
the nucleotide sequence encodes a promoter that is operably linked to the sequence encoding the interfering RNA such that the promoter can mediate transcription of the interfering RNA in neurons;
the promoter is a human methyl CpG binding protein 2 (MECP2) promoter;
the AAV vector is administered intranasally;
at least 200 billion particles of the AAV vector are administered to the subject;
the AAV vector is administered in an amount sufficient to achieve a multiplicity of infection (MOI) per target cell of at least 3;
the target cells are neurons of the brain;
the administering transfects the neurons with the AAV9 vector;
transfection of the neurons with the AAV9 vector causes expression of the interfering RNA;
expression of the interfering RNA reduces the concentration of the RNA transcript in the neurons;
the reduction of the concentration of the RNA transcript in the neurons reduces expression of the human 5HT-2A receptor in the brain of the subject by at least 20 percent; and
reduced expression of the human 5HT-2A receptor in the brain of the subject treats the symptoms of anxiety or depression.

2. A method to modulate brain activity in a brain of a subject, comprising:
providing an adeno-associated virus (AAV) vector; and
administering the AAV vector to the brain of the subject,
wherein:
the AAV vector comprises a nucleic acid that comprises a nucleotide sequence that encodes an interfering RNA;
the interfering RNA comprises a reverse complement sequence that is a reverse complement of an RNA transcript that encodes a human neurotransmitter receptor;
the administering transfects neurons of the brain with the AAV vector;
transfection of the neurons with the AAV vector causes expression of the interfering RNA;
expression of the interfering RNA reduces the concentration of the RNA transcript in the neurons;
the reduction of the concentration of the RNA transcript in the neurons reduces expression of the human neurotransmitter receptor in the brain; and
reduced expression of the human neurotransmitter receptor in the brain of the subject modulates the brain activity.

3. The method as claimed in claim 2, wherein the AAV vector is an AAV serotype 9 (AAV9) vector.

4. The method as claimed in claim 2, wherein the human neurotransmitter receptor is a G protein-coupled receptor.

5. The method as claimed in claim 4, wherein:
binding of a neurotransmitter to the human neurotransmitter receptor activates phospholipase C to cause calcium release; and
the administering modulates brain activity in the subject by modulating neurotransmitter-dependent calcium release in neurons that are transfected with the AAV vector.

6. The method as claimed in claim 2, wherein the human neurotransmitter receptor is 5-hydroxytryptamine receptor 2A (5HT-2A receptor).

7. The method as claimed in claim 4, wherein:
binding of a neurotransmitter to the human neurotransmitter receptor inhibits adenylyl cyclase to decrease cAMP; and
the administering modulates brain activity in the subject by modulating cAMP concentration in neurons that are transfected with the AAV vector.

8. The method as claimed in claim 2, wherein the human neurotransmitter receptor is 5-hydroxytryptamine receptor 1A (5HT-1A receptor).

9. The method as claimed in claim 2, wherein the interfering RNA comprises a nucleotide sequence selected from:

| SEQ ID NO. 5 | |
|---|---|
| | (UUCUGAAGACAAAGAACUCUG); |
| | SEQ ID NO. 7 |
| | (CAGAGUUCU); |
| | SEQ ID NO. 8 |
| | (UGUCUUCAGAA); |
| | SEQ ID NO. 9 |
| | (CUGUUCUGAAGACAAAGAACUCUGGU); |
| SEQ ID NO. 10 | |
| | (ACCAGAGUUCUGUCUUCAGAACAG); |
| | and |
| SEQ ID NO. 11 | |
| | (GUUUUGGCCACUGACUGAC). |

10. The method as claimed in claim 2, wherein the interfering RNA has at least 95 percent sequence identity with at least 20 consecutive nucleotides set forth in
(SEQ ID NO. 4)
GCUGUUCUGAAGACAAAGAACUCUGGUUUUGGCCACUGACUGACCAGAGU
UCUGUCUUCAGAACAG

11. The method as claimed in claim 2, wherein:
the nucleotide sequence encodes a promoter that is operably linked to the sequence encoding the interfering RNA such that the promoter can mediate transcription of the interfering RNA in neurons; and
the promoter is a human methyl CpG binding protein 2 (MECP2) promoter or a human alpha-synuclein promoter.

12. The method as claimed in claim 2, comprising administering the AAV vector in an amount sufficient to achieve a multiplicity of infection (MOI) per neuron of at least 3, wherein the AAV vector is administered intranasally.

13. The method as claimed in claim 1, comprising administering an olfactory test to the subject prior to administering the AAV vector, wherein the AAV vector is administered intranasally.

14. The method as claimed in claim 1, comprising focusing pulsed ultrasound in regions of the brain, wherein the regions of the brain in which the pulsed ultrasound is focused display reduced expression of the human neurotransmitter receptor per neuron relative to other regions of the brain in which the pulsed ultrasound is not focused.

15. The method as claimed in claim 1, comprising focusing repetitive Transcranial Magnetic Stimulation in regions of the brain, wherein the regions of the brain in which the repetitive Transcranial Magnetic Stimulation is focused display reduced expression of the human neurotransmitter receptor per neuron relative to other regions of the brain in which the repetitive Transcranial Magnetic Stimulation is not focused.

16. The method as claimed in claim 14, wherein the region comprises at least a portion of one or more of the amygdala, dorsal amygdala, hippocampus, hypothalamus, interpeduncular nucleus, anterior cingulate cortex, dorsal anterior cingulate cortex, posterior cingulate cortex, prefrontal cortex, ventromedial prefrontal cortex, pons, pontine nuclei, cerebellum, Purkinje layer of the cerebellum, orbital gyrus, and caudate nucleus.

17. The method as claimed in claim 1, comprising readministering the AAV vector to the subject at least 6 months and up to 12 months following the administering.

18. A method to treat a mental health condition in a subject, comprising:
determining that the subject presents with the mental health condition; and
performing the method as claimed in claim 1,
wherein:
the mental health condition is stress, anxiety, depression, attention deficit disorder (ADD), obsessive-compulsive disorder (OCD), post-traumatic stress disorder (PTSD), bipolar disorder, a personality disorder, memory concern, dementia, or inattention.

19. A recombinant nucleic acid that encodes an interfering RNA that has at least 95 percent sequence identity with at least 20 consecutive nucleotides set forth in
(SEQ ID NO. 4)
GCUGUUCUGAAGACAAAGAACUCUGGUUUUGGCCACUGACUGACCAGAGU
UCUGUCUUCAGAACAG

20. A recombinant nucleic acid that encodes an interfering RNA that comprises a nucleotide sequence selected from:

| SEQ ID NO. 5 | |
|---|---|
| | (UUCUGAAGACAAAGAACUCUG); |
| SEQ ID NO. 7 | |
| | (CAGAGUUCU); |
| SEQ ID NO. 8 | |
| | (UGUCUUCAGAA); |
| SEQ ID NO. 9 | |
| | (CUGUUCUGAAGACAAAGAACUCUGGU); |
| SEQ ID NO. 10 | |
| | (ACCAGAGUUCUGUCUUCAGAACAG); |
| | and |
| SEQ ID NO. 11 | |
| | (GUUUUGGCCACUGACUGAC). |

21. The recombinant nucleic acid as claimed in claim 19, wherein the recombinant nucleic acid is RNA.

22. The recombinant nucleic acid as claimed in claim 19, wherein:
the nucleic acid is a short hairpin RNA (shRNA);
the shRNA comprises a first sequence, a second sequence, and a third sequence;
the first sequence and the third sequence form intramolecular base pair interactions that hybridize the first sequence to the third sequence;
the second sequence is juxtaposed between the first sequence and the third sequence; and
the second sequence comprises a hairpin turn that lacks intramolecular base pair interactions.

23. The recombinant nucleic acid as claimed in claim 19, wherein the recombinant nucleic acid is single-stranded DNA.

24. The recombinant nucleic acid as claimed in claim 19, comprising a promoter that is operably linked to a sequence encoding the interfering RNA such that the promoter can mediate transcription of the interfering RNA in neurons, wherein the promoter is a human methyl CpG binding protein 2 (MECP2) promoter or an alpha-synuclein promoter.

25. An adeno-associated virus comprising the recombinant nucleic acid of claim 19.


SEQUENCE LISTING (13 sequences)

SEQ ID NO: 1 - DNA, 23 bp, synthetic construct
  source 1..23: mol_type = other DNA
tgcaattaggtgacgactcgagg

SEQ ID NO: 2 - DNA, 200 bp, synthetic construct
  source 1..200: mol_type = other DNA
gaagacaatatctccctgagctcaattccaaactccttaatgcaattaggtgactagtgaattcaacttgtttattgcagcttataatggttacaaataaagcaatagcatcacaaatttcacaaataaagcatttttttcactgctcgaggctctaccctaatgacttcaactccagggatgctaacacttccgaagcc

SEQ ID NO: 3 - RNA, 66 nt, synthetic construct
  source 1..66: mol_type = other RNA
gctgagcacatccaggtaaatccaggttttggccactgactgacctggatttctggatgtgctcag

SEQ ID NO: 4 - RNA, 66 nt, synthetic construct
  source 1..66: mol_type = other RNA
gctgttctgaagacaaagaactctggttttggccactgactgaccagagttctgtcttcagaacag

SEQ ID NO: 5 - RNA, 21 nt, synthetic construct
  source 1..21: mol_type = other RNA
ttctgaagacaaagaactctg

SEQ ID NO: 6 - RNA, 21 nt, Homo sapiens
  source 1..21: mol_type = other RNA
cagagttctttgtcttcagaa

SEQ ID NO: 7 - skipped

SEQ ID NO: 8 - RNA, 11 nt, Homo sapiens
  source 1..11: mol_type = other RNA
tgtcttcagaa

SEQ ID NO: 9 - RNA, 26 nt, synthetic construct
  source 1..26: mol_type = other RNA
ctgttctgaagacaaagaactctggt

SEQ ID NO: 10 - RNA, 24 nt, synthetic construct
  source 1..24: mol_type = other RNA
accagagttctgtcttcagaacag

SEQ ID NO: 11 - RNA, 19 nt, Musca sp.
  source 1..19: mol_type = other RNA
gttttggccactgactgac

SEQ ID NO: 12 - DNA, 4462 bp, synthetic construct
  source 1..4462: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaaggcaattgagcttagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccgggctagcgtttaaacttaagcttggtaccgagctcggatcccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttatcgcggccgcgaattcgttgcttatcccttatcctgtctggctgctggaggcttgctgaaggctgtatgctgagcacatccaggtaaatccaggttttggccactgactgacctggatttctggatgtgctcaggatacagtgcctgttactagcattcacgtggaacaaatggccgccgccgtgggaggatgacaagaccatgagtcactctgctggatgaacgtagaagtcaggctctatcacttcttaatgtgctcatcgtaacccggttactaggacgatcgatatcgtcgacggccgctcgagactctagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 4462 bp, synthetic construct
  source 1..4462: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaaggcaattgagcttagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccgggctagcgtttaaacttaagcttggtaccgagctcggatcccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttatcgcggccgcgaattcgttgcttatcccttatcctgtctggctgctggaggcttgctgaaggctgtatgctgttctgaagacaaagaactctggttttggccactgactgaccagagttctgtcttcagaacaggatacagtgcctgttactagcattcacgtggaacaaatggccgccgccgtgggaggatgacaagaccatgagtcactctgctggatgaacgtagaagtcaggctctatcacttcttaatgtgctcatcgtaacccggttactaggacgatcgatatcgtcgacggccgctcgagactctagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt